SEQUENCE LISTING (34 sequences)

SEQ ID NO: 1 - DNA, 101 bp, Human
ttgctttgaaatcttagaagagagcccactaattcaaggactcttactgtrggagcaactgctggttctatcacgtatgtactctttgttctttttctccc

SEQ ID NO: 2 - DNA, 101 bp, Human
gtacattacaagaaatcccactttcacgtctgggtaaatatcatttccttyctgtctccccaaaagcaatttgaaggtaaacaacaataacaaaggaatgt

SEQ ID NO: 3 - DNA, 101 bp, Human
ataagtgattacatatgaaaggccgtaaaacaaaattagaatgaaataagrtaataatttctccagggatttcaggtaactcaaaataatggtttggaaag

SEQ ID NO: 4 - DNA, 101 bp, Human
gtgggatgtcttatggctaccccagccacctgccttccccatgagtagacrgggagtcagtagaggggcttcacgccagcccaccagagccacgagaacat

SEQ ID NO: 5 - DNA, 101 bp, Human
ctgaaaacccacctggttccggaatcctgtcctcagcttcttaatataacygccttaaaactttaatcccacttgcccctgttacctaattagagcagatg

SEQ ID NO: 6 - DNA, 101 bp, Human
ggcatggggctgaagctgatttattattcagttggtgctggtatcactgayaaaggagaagtcccgaatggctacaacgtctccagatcaaccacagagga

SEQ ID NO: 7 - DNA, 101 bp, Human
tatcgtaagaagctggaagaagagctcaagtttttggtttactttcagaaygaagaacttattcagaaagcagaaataatcaatgagcgatttttagccca

SEQ ID NO: 8 - DNA, 101 bp, Human
tatttaaacgtaggatagtagtaaggagaatcttaaatcttagaaacttgrgggtatgacaagagcaattcctaaatccagatgatgattttaccattgct

SEQ ID NO: 9 - DNA, 101 bp, Human
atagtgacacacaagagagatgaagtggatacaaaaataaacttaaacctrgtaataaactgaacacgtaaatcctatagcttaaactagctcttaaaata

SEQ ID NO: 10 - DNA, 101 bp, Human
tcttctatttctccagagaaaaaatccatatacctgaaagatctgatgaarcccagcgtgtttttaaaagttcgaagacatcttcatgcgacaaaagtgat

SEQ ID NO: 11 - DNA, 101 bp, Human
tccaagctctgccacttagtagctctgtcgatcttgggtaaattacttcayctctctgggctttagtgtccacatcttaaaatgggaaataacaacaagac

SEQ ID NO: 12 - DNA, 101 bp, Human
atttggccgggggcagctgaggctcaggttacctcaattcttgagtttctraagaggcatatccaggggtgggttacgagaacaagagcctgcccagtctg

SEQ ID NO: 13 - DNA, 101 bp, Human
ttcagaaccacggtctggctcctgaagcagccctctcaagcagtcatcctkctctcagtcagaaactgctttacttctgcaacatctagaataaattacca

SEQ ID NO: 14 - DNA, 101 bp, Human
caccacataccaaaatcagtgagaaatattggattctttttggctgggaaygaatagttcggtggggaaagaccctattattgggaggcccagacaagtga

SEQ ID NO: 15 - DNA, 101 bp, Human
ttttagtagatactgaatatttcaccatgaaccaccaagttaccatgtacyttgaggtgtccattgaaaactaatgctttctgtaattgctttatgttctt

SEQ ID NO: 16 - DNA, 101 bp, Human
ggaacccaggcaaggggcatttgctgactacagtgtgtctttcagcaaacrcagccggaggccccccacccatggaagggacagccttgccaagaaaactg

SEQ ID NO: 17 - DNA, 101 bp, Human
tccaacgaaagccatgaccacatggatgatatggatgatgaagatgatgaygaccatgtggacagccaggactccattgactcgaacgactctgatgatgt

SEQ ID NO: 18 - DNA, 101 bp, Human
cactgagtcactgagtcttcaaagttcctgcttattcattacagatcttayctcctttccctcatccatggaaggatgttatttataaagtgttttattga

SEQ ID NO: 19 - DNA, 101 bp, Human
ctggcagcccctggtcctggtgctcctggtgctgggctgctgctttgctgyccccagacagcgccagtccacccttgtgctcttccctggagacctgagaa

SEQ ID NO: 20 - DNA, 101 bp, Human
ccggatgttccccggggtgcctttggacacgcacgacgtcttccagtaccraggtgagggctgaggaggatcccttcgtgagacaccacactaagctcctc

SEQ ID NO: 21 - DNA, 101 bp, Human
acgcccatttcgacgatgacgagttgtggtccctgggcaagggcgtcggtkagattctgagtcctcctggcccctgattcccttcattctctcccactcat

SEQ ID NO: 22 - DNA, 101 bp, Human
gagtgctggttctcctcgcggtgctgcctgtgctcctcctgcagatcactsttggcctcgtcttcctctgcctgcagtacagactgagaggtacagggcag

SEQ ID NO: 23 - DNA, 997 bp, Human
atgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagcgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaataaaggccccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctag

SEQ ID NO: 24 - DNA, 561 bp, Human
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtaccctggctaaagccgggccggagccctctgccctctcatgcccgcagccagcctgggctgtgcaacatgtacaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaagggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctga

SEQ ID NO: 25 - DNA, 930 bp, Human
atgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagttcgacagggttgccctatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagttggtgggtgaatgggaaggaggtgcacagtggggtcagcacagacccgcagcccctcaaggagcagcccgccctcaatgactccagatactgcctgagcagccgcctgagggtctcggccaccttctggcagaacccccscaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 26 - DNA, 1009 bp, Human
atgctgggcatctggaccctcctacctctggttcttacgtctgttgctagattatcgtccaaaagtgttaatgcccaagtgactgacatcaactccaagggattggaatttgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattctgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtgcctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 27 - DNA, 972 bp, Human
atgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaa

SEQ ID NO: 28 - DNA, 1707 bp, Human
atgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattgtatatgtgtcacgttgacagtcataattgtgtgttctgttttcatctataaaatcttcaagattgacattgtgctttggtacagggattcctgctatgattttctcccaataaaagcttcagatggaaagacctatgaccatatatactgtatccaaagactgttggggaagggtctacctctgactgtgatatttttgtgtttaaagtcttgcctgaggtcttggaaaaacagtgtggatataagctgttcatttatggaagggatgactacgttgggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtag

SEQ ID NO: 29 - DNA, 867 bp, Human
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 30 - DNA, 275 bp, Human
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggatacatcaactctttggagatgagctag

SEQ ID NO: 31 - DNA, 2121 bp, Human
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggcgcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggacattgggaaccagctgtatttgttcaaggatgggaagtactggcgattctctgagggcagggggagccggccgcagggccccttccttatcgccgacaagtggcccgcgctgccccgcaagctggactcggtctttgaggaccgctctccaagaagcttttcttcttctctgggcgccaggtgtgggtgtacacaggcgcgtcggtgctcgcccgaggcgtctggacaagctgggcctgggagccgacgtggcccaggtgaccggggccctccggagtccagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 32 - DNA, 757 bp, Human
atggcaagcttatcgagaccctctctgcccagctgcctctgctccttcctcctcctcctcctcctccaagtgtcttccagctatgcagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaagatcctttctactgggtgagccctggagtgctggttctcctcgcggtgctgcctgtgctcctcctgcagatcactgttggcctcgtcttcctctgcctgcagtacagactgagaggaaaacttcgagcagagatagagaatctccaccggacttttgatccccactttctgagggtgccctgctggaagataaccctgtttgtaattgtgccggttcttggacccttggttgcttgatcatctgctacaactggctacatcgaagactagcagggcaattccttgaagagctactcttccacctggaagccctctctggctaa

SEQ ID NO: 33 - DNA, 693 bp, Human
atggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaa

SEQ ID NO: 34 - DNA, 2589 bp, Human
atggcacatacttttagaggatgctcattggcatttatgtttataatcacgtggctgttgattaaagcaaaaatagatgcgtgcaagagaggcgatgtgactgtgaagccttcccatgtaattttacttggatccactgtcaatattacatgctctttgaagcccagacaaggctgctttcactattccagacgtaacaagttaatcctgtacaagtttgacagaagaatcaattttcaccatggccactccctcaattctcaagtcacaggtcttccccttggtacaaccttgtttgtctgcaaactggcctgtattaatagtgatgaaattcaaatatgtggagcagagatcttcgttggtgttgctccagaacagcctcaaaatctatcctgcatacagaagggagaacaggggactgtggcctgcacctgggaaagaggacgagacacccacttatacactgagtatactctacagctaagtggaccaaaaaatttaacctggcagaagcaatgtaaagacatctattgtgactatttggactttggaatcaacctcacccctgaatcacctgaatccaatttcacagccaaggttactgctgtcaatagtcttggaagctcctcttcacttccatccacattcacattcttggacatagtgaggcctcttcctccgtgggacattagaatcaaatttcaaaaggcttctgtgagcagatgtaccctttattggagagatgagggactggtactgcttaatcgactcagatatcggcccagtaacagcaggctctggaatatggttaatgttacaaaggccaaaggaagacatgatttgctggatctgaaaccatttacagaatatgaatttcagatttcctctaagctacatctttataagggaagttggagtgattggagtgaatcattgagagcacaaacaccagaagaagagcctactgggatgttagatgtctggtacatgaaacggcacattgactacagtagacaacagatttctcttttctggaagaatctgagtgtctcagaggcaagaggaaaaattctccactatcaggtgaccttgcaggagctgacaggagggaaagccatgacacagaacatcacaggacacacctcctggaccacagtcattcctagaaccggaaattgggctgtggctgtgtctgcagcaaattcaaaaggcagttctctgcccactcgtattaacataatgaacctgtgtgaggcagggttgctggctcctcgccaggtctctgcaaactcagagggcatggacaacattctggtgacttggcagcctcccaggaaagatccctctgctgttcaggagtacgtggtggaatggagagagctccatccagggggtgacacacaggtccctctaaactggctacggagtcgaccctacaatgtgtctgctctgatttcagagaacataaaatcctacatctgttatgaaatccgtgtgtatgcactctcaggggatcaaggaggatgcagctccatcctgggtaactctaagcacaaagcaccactgagtggcccccacattaatgccatcacagaggaaaaggggagcattttaatttcatggaacagcattccagtccaggagcaaatgggctgcctcctccattataggatatactggaaggaacgggactccaactcccagcctcagctctgtgaaattccctacagagtctcccaaaattcacatccaataaacagcctgcagccccgagtgacatatgtcctcgtggatgacagctctgacagctgctggtgaaagttcccacggaaatgagagggaattttgtctgcaaggtaaagccaattggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttgcacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgaggcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

(19) United States
(12) Patent Application Publication
(10) Pub. No.: US 2010/0285600 A1
(43) Pub. Date: Nov. 11, 2010

(54) Markers associated with the therapeutic efficacy of glatiramer acetate

(75) Inventors: Doron Lancet, Tel Aviv (IL); Jacques Beckmann, Rehovot (IL); Nili Avidan, Rehovot (IL); Edna Ben-Asher, Tel Aviv (IL); Clara Singer, Rehovot (IL); Liat Hayardeny, Tel Aviv (IL); Dan Goldstaub, Even Yehudah (IL); Iris Grossman, Haifa (IL); Ariel Miller, Haifa (IL)

Correspondence Address: COOPER & DUNHAM, LLP, 30 Rockefeller Plaza, 20th Floor, NEW YORK, NY, 10112, US

(73) Assignee: Teva Pharmaceutical Industries, Ltd.

(21) Appl. No.: 12/660,258

(22) Filed: Feb. 22, 2010

Related U.S. Application Data
(63) Continuation of application No. 11/409,590, filed on Apr. 24, 2006, now abandoned.
(60) Provisional application No. 60/674,545, filed on Apr. 25, 2005.

(51) Int. Cl. G01N 33/50

(52) U.S. Cl. 436/94

(57) ABSTRACT

The present invention is directed to methods and kits based, at least in part, on the identification of allele-specific responsiveness or non-responsiveness to glatiramer acetate for the treatment of autoimmune disorders, such as relapsing-remitting multiple sclerosis. The allele-specific responsiveness or non-responsiveness is based on polymorphisms in the following genes, CTSS, MBP, TCRB, CD95, CD86, IL-1R1, CD80, SCYA5, MMP9, MOG, SPP1 and IL-12RB2.

### RELATED APPLICATIONS

This application claims priority under 35 U.S.C. §119(e) to U.S. Provisional Application Ser. No. 60/674,545 filed on Apr. 25, 2005, the entirety of which is incorporated herein by reference.

### BACKGROUND

Multiple sclerosis (MS) is the most common neurological disease of young adults, afflicting worldwide approximately one million individuals. Pugliatti M. et al., Clin Neurol Neurosurg, 104(3):182-91 (2002). MS can be divided into a number of clinical sub-types, with most patients suffering from the most prevalent type (afflicting about 85-90% of patients at onset of disease), classified as relapsing-remitting (RR-MS). Noseworthy J H, et al., N Engl J Med., 343(13):938-52 (2000); Hafler D. A., J Clin Invest., 113(6):788-94 (2004).

Several medications have been approved and clinically ascertained as efficacious for the treatment of RR-MS; including BETASERON®, AVONEX® and REBIF®, which are derivatives of the cytokine interferon beta (IFNB), whose mechanism of action in MS is generally attributed to its immunomodulatory effects, antagonizing pro-inflammatory reactions and inducing suppressor cells. Revel M. Pharmacol Ther., 100(1):49-62 (2003). COPAXONE® (glatiramer acetate) follows presumably a distinct mode-of-action. Wolinsky I S, Expert Opin Pharmacother., 5(4):875-91 (2004). Glatiramer acetate (“GA”) is by design a mixture of synthetic polypeptides aimed at mimicking the amino acid composition of myelin basic protein (MBP), which is considered to be the primary autoantigen targeted in this disease. Neuhaus 0. et al., Neurology, 56(6):702-8 (2001). Independently conducted trials of GA treatment reaffirm its effectiveness in reducing relapse rate and CNS activity. Wolinsky J S, Expert Opin Pharmacother., 5(4):875-91 (2004); Rovaris M, et al., AJNR Am J. Neuroradiol., 24(1):75-81 (2003); Rovaris M, et al., Neurology, 59(9): 1429-32 (2002).

The mechanism by which GA induces its beneficial effect has been extensively investigated, and these studies demonstrate that GA exerts its therapeutic activity by immunomodulating various levels of the immune response, which differ in their degree of specificity. R. Amon and R. Aharoni, PNAS, 101(Supp.2):14593-14598 (2004). The prerequisite step is the binding of GA to MHC class II molecules; GA exhibits a very rapid, high, and efficient binding to various MHC class II molecules on murine and human antigen-presenting cells, and even displaced peptides from the MHC-binding site. M. Fridkis-Hareli et al., PNAS USA, 9:4872-76 (1994). This competition for binding to the MHC can consequently lead to inhibition of various pathological effector functions. It has been demonstrated that GA promotes T helper 2 (Th2) cell development and increased IL-10 production through modulation of dendritic cells. P. L. Vieira et al., J. Immunol., 178:4483-84 (2003). Duda, et al., J. Clin. Invest., 105(7):967-76 (2000).

The mode of action of GA is believed to be by initial strong promiscuous binding to MHC molecules and consequent competition with various myelin antigens for their presentation to T cells. R. Amon and R. Aharoni, PNAS, 101(Supp.2):14593-14598 (2004). A further aspect of its action is potent induction of specific suppressor cells of the Th2 type that migrate to the brain and lead to in situ by stander suppression. Furthermore, the GA-specific cells in the brain express the anti-inflammatory cytokines IL-10 and transforming growth factor β, in addition to brain-derived neurotrophic factor, whereas they do not express IFN-γ.

GA has been shown to be effective for treating conditions that result from activation of inflammatory T-cells, including prevention of graft rejection and amelioration of inflammatory bowel diseases. R. Amon and R. Aharoni, PNAS, 101(Supp.2):14593-14598 (2004). GA was effective in amelioration of graft rejection in two systems by prolongation of skin graft survival and inhibition of functional deterioration of thyroid grafts, across minor and major histocompatibility barriers. In transplantation systems GA treatment inhibited the detrimental secretion of Th1 inflammatory cytokines and induced beneficial Th2/3 anti-inflammatory response and GA has been shown to reduce macroscopic colonic damage, such as severe ulceration and inflammation in murine models resembling inflammatory bowel disease. R. Amon and R. Aharoni, PNAS, 101(Supp.2):14593-14598 (2004); Gur, et al., Clin. Immunol., 118:307-316 (2006). GA has been shown to suppress local lymphocyte proliferations and tumor necrosis factor-α detrimental secretion by induced transforming grown factor β, thus confirming the involvement of Th1 to Th2 shift in GA mode of action. R. Amon and R. Aharoni, PNAS, 101(Supp.2):14593-14598 (2004).

There is significant variability in the responses of patients to drugs; a patient that is non-responsive to a first treatment may be responsive to another treatment. For example, despite extensive research on MS, it is not known which of the available drugs will efficiently and safely arrest the progression of the disease in any given patient. The lack of objective tools that can assign risk-benefit profiles per medication per patient, dictates a mostly arbitrary prescription of drugs, via the “trial and error” paradigm.

However, personalized medicine, as predicted by pharmacogenetics (PGx), offers patients individually-tailored treatment programs. Pharmacogenetics can identify how well a patient will responds to a given treatment program, and thus provide safer and more effective treatment management.

### SUMMARY OF THE INVENTION

The present invention is based on the identification of genetic markers that are predictive of the effectiveness of glatiramer acetate (GA) in a subject. Specifically, the present invention is based, at least in part, on the identification of polymorphic nucleotides, corresponding to position 51 of polymorphic region sequences (SEQ ID NO: 1-22) of GA-responsive genes, that permit the responsiveness or non-responsiveness of a subject to glatiramer acetate to be accurately predicted. GA-responsive genes or genetic regions include, but are not limited to, Cathepsin S (CTSS), Myelin basic protein (MBP), T-cell receptor β (TCRB or TRBα), Apoptosis antigen 1 (CD95 or FAS), CD86, Interleukin-1 receptor 1 (IL-IR1), CD80, Chemokine ligand 5 (CCL5 or SCYAS), Matrix metalloproteinase-9 (MMP9), Myelin oligodendrocyte glycoprotein (MOG), Osteopontin (SPP1) and Interleukin-12 receptor β 2 (IL-12RB2) (hereinafter also referred to as GA-responsive genes).

In a first aspect, the present invention comprises a method for identifying a likely responder or non-responder to treatment with glatiramer acetate. The method includes the steps of obtaining a nucleic acid sample from a subject having symptoms associated with an autoimmune disorder that is amenable to treatment with GA, and determining the genetic profile of the subject in one or more GA-responsive genes. The GA-responsive genes include CTSS, MBP, TCRB, CD95, CD86, IL-1R1, CD80, SCYA5, MMP9, MOG, SPP1 and IL-12RB2. The genetic profile can be ascertained by determining the presence of a polymorphic marker or nucleotide in the sample. The polymorphic marker is located at a region corresponding to position 51 of one or more of SEQ ID No's: 1, 2, 3, 4, 5, 6, 7, 8, 9, 10, 11, 12, 14, 15, 16, 17, 18, 20, 21 and 22 or the complements thereof. The genetic profile also may be ascertained by determining the presence of a polymorphic marker that is in linkage disequilibrium with the polymorphic marker located at the region corresponding to position 51 of one or more of SEQ ID NO′S: 1, 2, 3, 4, 5, 6, 7, 8, 9, 10, 11, 12, 14, 15, 16, 17, 18, 20, 21 and 22 or the complements thereof.

In one preferred embodiment, the subject is determined to be a responder to glatiramer acetate treatment when the polymorphic marker located at the region corresponding to position 51 is a guanine of SEQ ID NO:2, an adenine of SEQ ID NO:3, an adenine of SEQ ID NO:4, a cytidine of SEQ ID NO:6, a guanine of SEQ ID NO:9, a thymidine of SEQ ID NO:11, an adenine of SEQ ID NO: 12, a guanine of SEQ ID NO: 16, and a thymidine of SEQ ID NO:18, or the complements thereof.

In other preferred embodiments, the subject is determined to be a non-responder to glatiramer acetate when the polymorphic marker located at the region corresponding to position 51 is a guanine of SEQ ID NO: 10, a thymidine of SEQ ID NO: 14, an adenine of SEQ ID NO:20, a thymidine of SEQ ID NO:21, and a cytidine of SEQ ID NO:22, or the complements thereof. The polymorphic marker can be determined on one or both genomic copies. The markers of the invention may be assessed, singly or in combination in the methods described herein.

Diseases and/or conditions amenable to treatment with GA include autoimmune disorders resulting from activation of inflammatory T-cells, and/or an imbalance between pro-inflammatory and anti-inflammatory reactivity. Such diseases and conditions include, for example, RR-MS, inflammatory bowel diseases such as Crohn's disease or colitis, and graft rejection.

In some embodiments, the genetic profile of the individual is determined by contacting the nucleic acid obtained from the subject with at least one probe or primer which hybridizes to the polymorphic marker or 5′ or 3′ to the polymorphic marker. In further embodiments, the probe or primer is capable of specifically hybridizing to the polymorphic marker or 5′ or 3′ to the polymorphic marker. The polymorphic marker is located at a region corresponding to position 51 of any one of SEQ ID Nos: 1-22 or the complements thereof. The polymorphic marker at position 51 may be any one of a guanine of SEQ ID NO:2, an adenine of SEQ ID NO:3, an adenine of SEQ ID NO:4, a cytidine of SEQ ID NO:6, a guanine of SEQ ID NO:9, a thymidine of SEQ ID NO:11, a guanine of SEQ ID NO:16, a thymidine of SEQ ID NO:18, a guanine of SEQ ID NO:10, an adenine of SEQ ID NO:12, a thymidine of SEQ ID NO:14, an adenine of SEQ ID NO:20, a thymidine of SEQ ID NO:21, and a cytidine of SEQ ID NO:22, a cytidine at position 51 of SEQ ID NO:1, a cytidine at position 51 of SEQ ID NO:2, an adenine at position 51 of SEQ ID NO:3, a guanine at position 51 of SEQ ID NO:4, a thymidine at position 51 of SEQ ID NO:5, an adenine at position 51 of SEQ ID NO:10, a thymidine at position 51 of SEQ ID NO:11, a guanine of at position 51 SEQ ID NO:8, an adenine at position 51 of SEQ ID NO:9, a guanine at position 51 of SEQ ID NO:15, an adenine at position 51 of SEQ ID NO:16, a cytidine at position 51 of SEQ ID NO:6 and a cytidine at position 51 of SEQ ID NO:7, or the complements thereof. The probe or primer can be labeled.

In some embodiments, the genetic profile is determined by the methods disclosed herein, including, allele specific hybridization, by primer specific extension, an oligonucleotide ligation assay or by single-stranded conformation polymorphism.

In another aspect, the invention relates to a method of identifying a responder to treatment with glatiramer acetate. The method includes the steps of obtaining a sample from a subject having symptoms associated with an autoimmune disorder that is amenable to treatment with GA, and determining the subject's genetic profile for CTSS. The genetic profile can be ascertained by determining the presence of polymorphic markers in the sample. The polymorphic markers are located at regions corresponding to position 51 of SEQ ID NO:1, position 51 of SEQ ID NO:2 and position 51 of SEQ ID NO:3, or the complements thereof. The presence of the polymorphic markers are indicative of a responder to glatiramer acetate. In a further embodiment, the polymorphic markers located at regions corresponding to position 51 are a cytidine of SEQ ID NO:1, a cytidine of SEQ ID NO:2 and an adenine of SEQ ID NO:3 or the complements thereof. The genetic profile can also be ascertained by determining the presence of one or more polymorphic markers which are in linkage disequilibrium with the polymorphic marker located at the region corresponding to position 51 of SEQ ID NO: 1, SEQ ID NO:2 and SEQ ID NO:3.

In another aspect, the invention relates to a method of identifying a likely non-responder to treatment with glatiramer acetate. The method includes the steps of obtaining a nucleic acid sample from a subject having symptoms associated with an autoimmune disorder that is amenable to treatment with GA, and determining the subject's genetic profile for MBP. The genetic profile can be ascertained by determining the presence of polymorphic markers in the sample. The polymorphic markers located at the regions corresponding to position 51 of SEQ ID NO: 4 and position 51 of SEQ ID NO: 5, or the complements thereof. The presence of the polymorphic markers are indicative of a non-responder to glatiramer acetate. In a further embodiment, the polymorphic markers located at the regions corresponding to position 51 are a guanine of SEQ ID NO: 4 and a thymidine of SEQ ID NO:5 or the complements thereof. The genetic profile can also be ascertained by determining the presence of one or more polymorphic markers which are in linkage disequilibrium with the polymorphic markers located at the regions corresponding to position 51 of SEQ ID NO:4 and position 51 of SEQ ID NO:5, or the complements thereof.

In another aspect, the invention relates to a method of identifying a likely non-responder to treatment with glatiramer acetate. The method includes the steps of obtaining a nucleic acid sample from a subject having symptoms associated with an autoimmune disorder that is amenable to treatment with GA, and determining the subject's genetic profile for CD86. The genetic profile can be ascertained by determining the presence of polymorphic markers in the sample. The polymorphic markers are located in the regions corresponding to position 51 of SEQ ID NO:10 and position 51 of SEQ ID NO:11 or the complements thereof. The presence of the polymorphic markers are indicative of a non-responder to glatiramer acetate. In another embodiment, the polymorphic markers corresponding to position 51 of SEQ ID NO:10 is an adenine and corresponding to position 51 of SEQ ID NO: 11 is a thymidine or the complements thereof. The genetic profile can also be ascertained by determining the presence of one or more polymorphic markers which are in linkage disequilibrium with the polymorphic markers located at regions corresponding to position 51 of SEQ ID NO:10 and position 51 of SEQ ID NO:11.

In another aspect, the invention relates to a method of identifying a likely responder to treatment with glatiramer acetate. The method includes the steps of obtaining a nucleic acid sample from a subject having symptoms associated with an autoimmune disorder that is amenable to treatment with GA, and determining the genetic profile of CD95. A genetic profile can be ascertained by determining the presence of polymorphic markers in the sample. The polymorphic markers are located at regions corresponding to position 51 SEQ ID NO:8 and position 51 of SEQ ID NO:9 or the complements thereof. The presence of the polymorphic markers are indicative of a responder to glatiramer acetate. In a further embodiment, the polymorphic markers located at the regions corresponding to position 51 of SEQ ID NO:8 is a guanine and at position 51 of SEQ ID NO:9 is an adenine or the complements thereof. The genetic profile can also be ascertained by determining the presence of one or more polymorphic markers which are in linkage disequilibrium with the polymorphic markers corresponding to position 51 SEQ ID NO:8 and position 51 of SEQ ID NO:9.

In another aspect, the invention relates to a method of identifying a likely responder to treatment with glatiramer acetate. The method includes the steps of obtaining a nucleic acid sample from a subject having symptoms associated with an autoimmune disorder that is amenable to treatment with GA, and determining the genetic profile of IL-12RB2. The genetic profile can be ascertained by determining the presence of polymorphic markers in the sample. The polymorphic markers are located in the regions corresponding to position 51 of SEQ ID NO:15 and position 51 of SEQ ID NO:16 or the complements thereof. The presence of the polymorphic markers are indicative of a responder to glatiramer acetate. In a further embodiment, the polymorphic markers located at the regions corresponding to position 51 of SEQ ID NO:15 is a guanine and at position 51 of SEQ ID NO:16 is an adenine or the complements thereof. The genetic profile can also be ascertained by determining the presence of one or more polymorphic markers which are in linkage disequilibrium with the polymorphic markers located at regions corresponding to position 51 of SEQ ID NO:15 and position 51 of SEQ ID NO: 16.

In another aspect, the invention relates to a method of identifying a likely non-responder to treatment with glatiramer acetate. The method includes the steps of obtaining a nucleic acid sample from a subject having symptoms associated with an autoimmune disorder that is amenable to treatment with GA, and determining the genetic profile of TCRB. A genetic profile can be ascertained by determining the presence of polymorphic markers in the sample. The polymorphic markers are located at regions corresponding to position 51 of SEQ ID NO:6 and position 51 of SEQ ID NO:7 or the complements thereof. The presence of the polymorphic markers are indicative of a non-responder to glatiramer acetate. In a further embodiment, the polymorphic markers located at the regions corresponding to position 51 of SEQ ID NO:6 is a cytidine and at position 51 of SEQ ID NO:7 is a cytidine or the complements thereof. The genetic profile can also be ascertained by determining the presence of one or more polymorphic markers which are in linkage disequilibrium with the polymorphic markers located at the regions corresponding to position 51 of SEQ ID NO:6 and position 51 of SEQ ID NO:7.

In another aspect, the invention relates to a kit comprising a primer or probe which detects or amplifies position 51 of the nucleic acid sequence selected from the group consisting of: SEQ ID NO:1, SEQ ID NO:2, SEQ ID NO:3, SEQ. ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, SEQ ID NO: 14, SEQ ID NO: 15, SEQ ID NO: 16, SEQ ID NO: 18, SEQ ID NO:20, SEQ ID NO:21, and SEQ ID NO:22, and packaging materials thereof. In a further embodiment the kit contains a detection means. Detection means include, hybridization of allele-specific oligonucleotides, sequence specific amplification, size analysis, sequencing, hybridization, nuclease digestion, single-stranded conformation polymorphism, primer specific extension, denaturing high performance liquid chromatography and an oligonucleotide ligation assay. In another embodiment the primer and/or probe selectively hybridize to a nucleotide selected from the group consisting of: a guanine at position 51 of SEQ ID NO:2, a adenine at position 51 of SEQ ID NO:3, a adenine at position 51 of SEQ ID NO:4, a cytidine at position 51 of SEQ ID NO:6, an guanine at position 51 of SEQ ID NO:9, a thymidine at position 51 of SEQ ID NO:11, a guanine at position 51 of SEQ ID NO:16, a thymidine at position 51 of SEQ ID NO:18, an adenine at position number 51 of SEQ ID NO:10, an adenine at position number 51 of SEQ ID NO:12, an thymidine at position 51 of SEQ ID NO:14, an adenine at position 51 of SEQ ID NO:20, an thymidine at position 51 of SEQ ID NO:21, cytidine at position 51 of SEQ ID NO:22, a cytidine at position 51 of SEQ ID NO:1, a cytidine at position 51 of SEQ ID NO:2, a guanine at position 51 of SEQ ID NO:4, a thymidine at position 51 of SEQ ID NO:5, an adenine at position 51 of SEQ ID NO:10, a guanine of at position 51 SEQ ID NO:8, an adenine at position 51 of SEQ ID NO:9, a guanine at position 51 of SEQ ID NO:15, a adenine at position 51 of SEQ ID NO:16, and a cytidine at position 51 of SEQ ID NO:7, or the complements thereof.

The nucleic acid molecules of the invention can be double- or single-stranded. Accordingly, in one embodiment of the invention, a complement of the nucleotide sequence is provided wherein the polymorphic marker has been identified. For example, where there has been a single nucleotide change from a thymidine to a cytidine in a single strand, the complement of that strand will contain a change from an adenine to a guanine at the corresponding nucleotide residue. The invention further provides allele-specific oligonucleotides that hybridize to the polymorphic markers or 5′ or 3′ to the polymorphic markers described herein.

In another preferred embodiment, the method comprises determining the nucleotide content of at least a portion of a GA-responsive gene, such as by sequence analysis. In yet another embodiment, determining the molecular structure of at least a portion of a GA-responsive gene is carried out by single-stranded conformation polymorphism (SSCP). In yet another embodiment, the method is an oligonucleotide ligation assay (OLA). Other methods within the scope of the invention for determining the molecular structure of at least a portion of a GA-responsive gene include hybridization of allele-specific oligonucleotides, sequence specific amplification, primer specific extension, and denaturing high performance liquid chromatography (DHPLC) and other methods known in the art. In at least some of the embodiments of the invention, the probe or primer is allele specific. Preferred probes or primers are single stranded nucleic acids, which optionally are labeled.

Other features and advantages of the invention will be apparent from the following detailed description and the claims.

### BRIEF DESCRIPTION OF THE DRAWINGS

FIGS. 1A-1J depict the responder and non-responder genotype distributions for CTSS, MBP, TCRB, CD86, CD80, CD95, and IL12R2 in GA and placebo treated cohorts. The European/Canadian MRI trial results are shown in FIGS. 1A-1H and the U.S. pivotal trial results are shown in FIGS. 11 and 1J. Each bar color represents carriers of a specific genotype, where black denotes homozygotes of the common allele, black and white stripes denote heterozygotes, and gray denotes homozygotes of the rare allele. Numbers of patients in each group are indicated above each bar. In each panel the Y axis shows percentage of positive or negative responders out of the total number of carriers of a specific genotype. The genotype displayed on the X axis can also be represented as the complement of that shown.¹-“combined” response definition; 2-“TI-lesion free” response definition; ³-“classical” response definition;

FIG. 2 depicts the haplotype distribution for genes in GA-treated and placebo-treated groups. The European/Canadian MM trial results are depicted in FIGS. 2A-E; the U.S. pivotal trial results are depicted in FIGS. 2F and 2G. Black bars denote responders and gray bars denote non-responders. Encoded haplotypes are shown on the Y axis, while their frequencies in the two treatment groups are shown on the X axis.

FIG. 3 depicts the nucleic acid sequences of the dbSNP ID′S described herein.

FIG. 4 depicts the open reading frames of the GA-responsive genes.

### DETAILED DESCRIPTION

The present invention is based, at least in part, on the identification of allele-specific responsiveness or non-responsiveness to glatiramer acetate for the treatment of an autoimmune disorder that is amenable to treatment with GA, in particular, for multiple sclerosis or Crohn's disease. The allele-specific responsiveness or non-responsiveness is based on polymorphisms in regions of CTSS, MBP, TCRB, CD95, CD86, IL-1R1, CD80, SCYAS, MMP9, MOG, SPP1 and IL-12RB2, herein referred to as GA-responsive genes.

The term “allele” refers to alternative forms of a gene or portions thereof. Alleles occupy the same locus or position on homologous chromosomes. When a subject has two identical alleles of a gene, the subject is said to be homozygous for the allele. When a subject has two different alleles of a gene, the subject is said to be heterozygous for the allele. Alleles of a specific gene, including the “GA responsive genes”, can differ from each other in a single nucleotide. An allele of a gene can also be a form of a gene containing one or more mutations or DNA sequence variants.

A “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term “nucleic acid,” and in particular “DNA molecule” or “RNA molecule,” refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). However, unless specifically stated otherwise, a designation of a nucleic acid includes both the non-transcribed strand referred to above, and its corresponding complementary strand. For purposes of clarity, when referring herein to a nucleotide of a nucleic acid, which can be DNA or an RNA, the terms “adenine”, “cytidine”, “guanine”, and “thymidine” and/or “A”, “C”, “G”, and “T”, respectively, are used. It is understood that if the nucleic acid is RNA, a nucleotide having a uracil base is uridine.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of one or more nucleotides or an insertion of one or more nucleotides relative to a reference allele. Typically, the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's of the invention correspond to position 51 of SEQ ID Nos: 1, 2, 3, 4, 5, 6, 7, 8, 9, 10, 11, 12, 14, 15, 16, 18, 20, 21 and 22.

SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon either directly (a “nonsense” SNP) or indirectly (by creating or abolishing a splice site). When a SNP does not alter the amino acid sequence of a protein, the SNP is usually “silent.” SNP's may also occur in noncoding regions of the nucleotide sequence. This may result in defective protein expression, e.g., as a result of alternative spicing, or changes in quantitative (spatial or temporal) expression patterns or it may have no effect.

The term “polymorphism” or “polymorphic” refers to the coexistence of more than one form of a gene or portion thereof. A portion of a gene in which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wild-type form. Other allelic forms are typically designated or alternative or variant alleles. Diploid organisms may be homozygous or heterozygous for allelic forms. A diallelic or biallelic polymorphism has two forms. A “polymorphic gene” refers to a gene having at least one polymorphic region.

The term “polymorphic nucleotide” or” polymorphic marker” refers to one or more nucleotides which can be used to determine whether an individual may or may not respond to GA treatment. The polymorphic marker may be a SNP. The polymorphic marker may correspond to position 51 of SEQ ID Nos: 1, 2, 3, 4, 5, 6, 7, 8, 9, 10, 11, 12, 14, 15, 16, 18, 20, 21 or 22 or an allele in linkage disequilibrium therewith. Polymorphic markers are described in Tables II and III, herein. Polymorphic marker also refers to the nucleotide that is complementary to the one stated. The term “genetic profile” refers to the information obtained from identification of the specific allelic variants of a subject. For example, a CTSS genetic profile refers to the specific allelic variants of a subject within the CTSS gene. For example, one can determine a subject's CTSS genetic profile by determining the identity of one or more of the nucleotides present at nucleotide residue 51 of SEQ ID NO:1 or corresponding nucleotide residue 118,433 of GI:21530888, nucleotide residue 51 of SEQ ID NO:2 or corresponding nucleotide residue 110,515 of GI:21530888 and nucleotide residue 51 of SEQ ID NO:3 or corresponding nucleotide residue 85,991 of GI:21530888 all of the CTSS gene, or the complements thereof. The genetic profile of a GA-responder or non-responder can be ascertained through identification of the identity of allelic variants in one or more genes which are associated with GA-responder or non-responder.

“GA” or “glatiramer acetate” refers to COPAXONE® (glatiramer acetate injection, Teva Pharmaceutical Industries Ltd.).

The term “relapsing-remitting multiple sclerosis” refers to multiple sclerosis characterized by clearly defined flare-ups or episodes of acute worsening of neurologic function, followed by partial or complete recovery periods (remissions).

The term “GA-responder” refers to a subject that is positively responsive, i.e. the patient's situation improves upon GA therapy. A “GA-responder” can be measured in any of multiple methods known in the art and disclosed herein. For example a “GA-responder” can be defined according to the criteria used in the European/Canadian MRI trial (Comi G, Filippi M, Wolinsky J S. European/Canadian multicenter, double-blind, randomized, placebo controlled study of the effects of glatiramer acetate on magnetic resonance imaging—measured disease activity and burden in patients with relapsing multiple sclerosis. European/Canadian glatiramer acetate Study Group. Ann Neurol.,;49(3):290-7 (2001)) and U.S. pivotal trial (Johnson K P, Brooks B R, Cohen J A, et al. Neurology, 45(7):1268-76 (1995); Johnson K P, Brooks B R, Cohen J A, et al. Neurology 50(3):701-8 (1998)) which are discussed herein.

As used herein, the term “GA-non-responder” is defined as a subject that does not adequately respond to GA-therapy. For example a “GA-non-responder” can be defined based on the criteria used in the European/Canadian MRI trial (Comi G, Filippi M, Wolinsky J S. European/Canadian multicenter, double-blind, randomized, placebo-controlled study of the effects of glatiramer acetate on magnetic resonance imaging—measured disease activity and burden in patients with relapsing multiple sclerosis. European/Canadian glatiramer acetate Study Group. Ann Neurol 2001; 49(3):290-7) and U.S. pivotal trial (Johnson K P, Brooks B R, Cohen J A, et al. Neurology 1995; 45(7):1268-76; Johnson K P, Brooks B R, Cohen J A, et al. Neurology 1998; 50(3):701-8) both of which are discussed herein.

The term “primer” (or “probe”) refers to a length of single-stranded nucleic acids, which is used in combination with a polymerase to amplify or extend a region from a template nucleic acid. Primers are generally short (e.g., 15-30 bases), but can be longer if required. The primer must contain a sequence which hybridizes with the template nucleic acid under the conditions used. Primers may be used singly, that is, a single primer consisting only of a single sequence can be used in the amplification reaction, and will produce one copy of one strand of the template per cycle of amplification. This can be done in situations where a large number of copies is not required, or where only one strand is to be copied (e.g., in producing antisense products), or if the sequence at the other end of the template is unsuitable for choosing a second primer. More generally, a pair of primers is used in an amplification reaction. The two are of different sequences, and are used in combination, and produce a copy of each template strand per cycle of amplification. The two different primers should not be complementary to each other, or they will hybridize to each other rather than the template, and the polymerase will then be unable to make a copy of the template. Commonly, the two primers are chosen from sequence at the 5′ end of each of the two complementary strands of the template nucleic acid. “Primer” also refers to a short nucleotide sequence complementary to the sequence of nucleotides 5′ or 3′ to the polymorphic nucleotide targeted for detection by an extension reaction. The “primer” is designed such that the polymorphic marker is detected by the methods disclosed herein.

The “primer” can be sequence specific which means a primer which specifically hybridizes with a nucleic acid sequence present in one or more alleles of a genetic locus or their complementary strands but not a nucleic acid sequence present in all the alleles of the locus. The sequence-specific primer does not hybridize with alleles of the genetic locus that do not contain the sequence polymorphism under the conditions used in the amplification method. For example a sequence specific primer would be a primer which specifically hybridizes with a cytidine corresponding to nucleotide position 51 of SEQ ID NO:6, but which does not hybridize with a thymidine corresponding to nucleotide position 51 of SEQ ID NO:6. The primer of the invention comprises a sequence that flanks and/or preferably overlaps, at least one polymorphic site occupied by any of the possible variant nucleotides. The nucleotide sequence of an overlapping probe can correspond to the coding sequence of the allele or to the complement of the coding sequence of the allele.

The term “hybridization probe” or “probe” as used herein is intended to include oligonucleotides which hybridize in a base-specific manner to a complementary strand of a target nucleic acid. Such probes include peptide nucleic acids, and described in Nielsen et al., (1991) Science 254: 1497-1 500. Probes can be any length suitable for specific hybridization to the target nucleic acid sequence. The most appropriate length of the probe may vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes can range form about 5 nucleotides to about 30 nucleotides in length. For example, probes can be 5, 6, 8, 10, 12, 14, 16, 18, 20, 22, 24, 25, 26, 28 or 30 nucleotides in length. The probe of the invention comprises a sequence that flanks and/or preferably overlaps, at least one polymorphic site occupied by any of the possible variant nucleotides. The nucleotide sequence of an overlapping probe can correspond to the coding sequence of the allele or to the complement of the coding sequence of the allele.

As used herein, the term “specifically hybridizes” or “specifically detects” or “specific hybridization” refers to the ability of a nucleic acid molecule of the invention to stably hybridize to either strand of a GA-responsive gene polymorphic region containing one allele but not to or less stably than a different allele under the same hybridization conditions. This selectivity is based on the nucleotide sequence of the probe, which is complementary to the target nucleic acid sequence or sequences.

A “haplotype” is a term denoting the collective allelic state of a number of closely linked polymorphic loci (i.e. SNPs) on a chromosome. This non-random association of alleles renders these markers tightly linked. Tight linkage (linkage disequilibrium, LD) can induce strong correlation between the genetic histories of neighboring polymorphisms and, when LD is very high, alleles of linked markers can sometimes be used as surrogates for the state of nearby loci. “Determining the subject's haplotype” refers to determining a subject's genetic profile or the unique chromosomal distribution of polymorphic nucleotides or polymorphic markers in or in the vicinity of a GA-responsive gene. For example, determining a subject's haplotype for MBP would require determining the nucleotides present in a subject's nucleic acid sample, on both his/her corresponding chromosomal regions, at a position corresponding to position 51 of SEQ ID NO:4 and at a position corresponding to position 51 of SEQ ID NO:5.

As used herein the term, “linkage disequilibrium” refers to co-inheritance of two or more alleles at frequencies greater than would be expected from the separate frequencies of occurrence of each allele in the corresponding control population. The expected frequency of occurrence of two or more alleles that are inherited independently is the population frequency of the first allele multiplied by the population frequency of the second allele. Alleles or polymorphisms that co-occur at expected frequencies are said to be in linkage equilibrium.

As used herein, the term “corresponding to” refers to a nucleotide in a first nucleic acid sequence that aligns with a given nucleotide in a reference nucleic acid sequence when the first nucleic acid and reference nucleic acid sequences are aligned. Alignment is performed by one of skill in the art using software designed for this purpose. As an example of nucleotides that “correspond,” the nucleotide at position 51 of SEQ ID NO:6 of TCRB “corresponds to” nucleotide position 27,091 of Gen Bank Accession # GI: 1552506 of TCRB, and vice versa.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul, Proc. Natl. Acad. Sci. USA 87:2264-2268 (1990), modified as in Karlin and Altschul, Proc. Natl. Acad. Sci. USA 90:5873-5877 (1993). Such an algorithm is incorporated into the NBLAST and XBLAST programs of Altschul, et al., J. Mol. Biol. 21 5:403-410 (1990). BLAST nucleotide searches can be performed With the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the 20 invention. To obtain gapped alignments for comparison purposes, Gapped BLAST can be utilized as described in Altschul et al., Nucleic Acids Res. 25:3389-3402 (1997). Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman, Proc. Natl. Acad. Sci. USA 85:2444-2448 (1988). When using the FASTA algorithm for comparing nucleotide or amino acid sequences, a PAM120 weight residue table can, for example, be used with a k-tuple value of 2.

The polymorphisms or markers described herein are single nucleotide polymorphisms (SNPs) at specific nucleotide residues within a GA-responsive gene. The GA-responsive genes include CTSS, MBP, TCRB, CD95, CD86, IL-1R1, CD80, SCYA5, MMP9, MOG, SPP1 and IL-12RB2 genes. The nucleotide sequences encoding the open reading frame for these genes are CTSS (SEQ ID NO:23), MBP (SEQ ID NO:24), TCRB (SEQ ID NO:25), CD95 (SEQ ID NO:26), CD86 (SEQ ID NO:27), IL-1R1 (SEQ ID NO:28), CD80 (SEQ ID NO:29), SCYA5 (SEQ ID NO:30), MMP9 (SEQ ID NO:31), MOG (SEQ ID NO:32), SSP1 (SEQ ID NO:33) and IL-12RB2 (SEQ ID NO:34). The GA-responsive genes have at least two alleles. One of the alleles for each GA-responsive gene is identified, herein, as being associated with a responsive or non-responsive phenotype. Similarly, each of the two alleles will either be considered a reference allele or a variant allele. The reference allele (i.e., the consensus sequence, or wild type allele) has been designated based on its frequency in a general U.S. Caucasian population sample. The reference allele is the more common of the two alleles; the variant is the less frequent of the two alleles. The allele corresponding to a responsive or non-responsive phenotype can be either a reference allele or a variant allele.

It is understood that the invention is not limited by the exemplified reference sequences, as variants of this sequence which differ at locations other than the SNP sites identified herein can also be utilized. The skilled artisan can readily determine the SNP sites in these other reference sequences which correspond to the SNP site identified herein by aligning the sequence of interest with the reference sequences specifically disclosed herein. Programs for performing such alignments are commercially available. For example, the ALIGN program in the GCG software package can be used, utilizing a PAM120 weight residue table, a gap length penalty of 12 and a gap penalty of 4, for example.

Two clinical trials, the European/Canadian trial and U.S. pivotal trial, were used to identify associations between GA responsiveness and SNPs in RR-MS patients. Caucasian patients, that had participated in one of two previously completed randomized, double blind, placebo-controlled, multi-centric clinical trials, were solicited to partake in the present study. In both clinical trials, patients were required to have a diagnosis of definite MS (Poser C M, et al. Ann Neurol., 13(3):227-31 (1983) and a relapsing-remitting course (Lublin F D and Reingold S C, Neurology 46(4):907-11 (1996). Ultimately, 73 and 101 DNA samples of Caucasian patients from the U.S. pivotal and European/Canadian MRI trials, respectively, were analyzed. GA dosage was consistent for both trials (i.e. daily 20-mg subcutaneous injection). Table I compares multiple variables between the general cohort study and the PGx cohort study for the European/Canadian trial and the U.S. pivotal trial.

The two clinical trials used different clinical end-points as described in the Examples. Twenty-seven candidate genes were selected based on their potential involvement in (a) GA's presumed mode-of-action; or (b) in MS pathogenesis; (c) representing general immune- and/or neurodegenerative-related molecules; or, (d) altered gene-expression profiles associated with MS. DNA was isolated from the 174 patients and genotyped for 63 SNPs according to previously described methods (Grossman I, et al., Genes Immun. (2004). A SNP-by-SNP and haplotype analysis identified SNPs correlating with a response and/or non-response to GA for CTSS, MBP, TCRB, CD95, CD86, IL-1R1, CD80, SCYA5, MMP9, MOG, SPP1 and IL-12RB2. Details describing the GA response definition and statistical analysis are described in the Examples.

The preferred polymorphic markers of the invention are listed in Table II and Table III. Table II corresponds to polymorphic markers determined by a SNPbySNP analysis. Table III corresponds to polymorphic markers determined by a haplotype analysis. Table II indicates the SEQ ID NO, in column 2, for the open reading frame for the GA-responsive genes. Column 4 identifies the NCBI database SNP identifier for each gene's polymorphic region, while column 5 identifies the SEQ ID NO or polymorphic region sequence for a sequence corresponding to the NCBI database SNP identifier. Each SEQ ID describing the SNP contains the polymorphic marker at position 51 and the flanking sequences. For example, nucleotide 51 of SEQ ID NO:17 is the polymorphic SNP position which corresponds to a cytidine or thymidine. Column 6 indicates the polymorphic marker which was identified in the present invention to correspond with a subject's response to GA treatment. Thus, a subject expressing the thymidine at position 51 of SEQ ID NO:17 is a likely GA responder. Column 7 indicates the polymorphic markers which are identified herein to be present in subjects who are unresponsive to GA treatment. Column 8 indicates the NCBI GenBank Accession GI number which identifies the nucleic acid sequence which contains the GA-responsive polymorphic region. The GI number identifies the nucleic acid sequence which is also referred to as the reference sequence. Column 9 indicates the nucleotide position in the GI sequence which corresponds to the polymorphic marker. For example nucleotide 27,091 of GI:1552506 corresponds to the polymorphic site (nucleotide 51) of SEQ ID NO:6.

The following polymorphisms have been found to correlate with GA-responders: thymidine at nucleotide 51 of SEQ ID NO:17 or the corresponding nucleotide 405 of GI:38146097, a cytidine at nucleotide 51 of SEQ ID NO: 6 or the corresponding nucleotide 214,464 of GI: 1552506, a thymidine at nucleotide 51 of SEQ ID NO:11 or the corresponding nucleotide, 112,096 of GI16572839, an adenine at position number 51 of SEQ ID NO:12 or the corresponding nucleotide 94,170 of GI: 19033951, a guanine at nucleotide 51 of SEQ ID NO:9 or the corresponding nucleotide, 163,560 of GI: 15384622, a guanine at nucleotide 51 of SEQ ID NO: 16 or the corresponding nucleotide, 103,042 of GI: 1 1990046, a thymidine at position 51 of SEQ ID NO:18 or the corresponding nucleotide 168,416, a guanine at nucleotide 51 of SEQ ID NO:2 or the corresponding nucleotide, 110,515, of GI:21530888, an adenine at position 51 of SEQ ID NO:3 or the corresponding nucleotide, 85,991 of GI:21530888 and an adenine at nucleotide 51 of SEQ ID NO:4 or the corresponding nucleotide, 128,344 of GI:27764783.

The following polymorphic nucleotides or markers were found to correlate with GA-non-responders: a guanine at position number 51 of SEQ ID NO:10 or the corresponding nucleotide 86,466 of GI:16572839, an thymidine at position 51 of SEQ ID NO:14 or the corresponding nucleotide 92,290 of GI:19033385, an adenine at position 51 of SEQ ID NO:20 or the corresponding nucleotide 2,022 of GI:4826835, an thymidine at position 51 of SEQ ID NO:21 or the corresponding nucleotide 669 at GI:4826835, and cytidine at position 51 of SEQ ID NO:22 or the corresponding nucleotide 673 at GI:455-45416.

Table III indicates the results obtained form the haplotype analysis as described in Example section. Column 2 indicates the SEQ ID NO for the open reading frame for the GA responsive genes identified based on a haploytpe analysis described herein. Column 4 identifies the NCBI database SNP identifier for each gene's polymorphic region, while column 5 identifies the SEQ ID NO or polymorphic region sequence for a sequence corresponding to the NCBI database SNP identifier. Each SNP SEQ ID contains the polymorphic marker at position 51. Columns 6 and 7 indicate the GA-responder and non-responder haplotype. A 0 indicates the presence of the frequent allele while a 1 indicates the presence of the rare allele. The order of the haploytpe code is identical to the order of the dbSNP IDS and the SNP SEQ ID NOS. Column 8 indicates the nucleotide or marker present at the polymorphic allele of nucleotide 51, which corresponds to either the GA-responder or -non-responder haploytpe. Column 9 indicates the NCBI GenBank Accession GI number which identifies the nucleic acid sequence which contains the GA-responsive polymorphic region. The GI number identifies the nucleic acid sequence which is also referred to as the reference sequence. Column 10 indicates the nucleotide position in the GI sequence which corresponds to the polymorphic marker. For example nucleotide 27,091 of GI:1552506 corresponds to the polymorphic site (nucleotide 51) of SEQ ID NO:6. The order of the dbSNP ID, SEQ ID NO for SNP, Haplotype nucleotide at position 51, Genbank Accession # and nucleotide position in the GenBank Accession number is identical to the order of the SNP SEQ ID NO.

In addition to the polymorphisms or alleles described herein, one of skill in the art can readily identify other polymorphic markers or alleles that are in linkage disequilibrium with a polymorphic marker or allele associated with GA-responders or GA-non-responders. For example, a nucleic acid sample from a first group of subjects who are GA-responders can be collected, as well as DNA from a second group of subjects who are GA-non-responders. The nucleic acid sample can then be compared to identify those alleles that are over-represented in the second group as compared with the first group, wherein such alleles are presumably associated with a GA-non-responder. Alternatively, alleles that are in linkage disequilibrium with an allele that is associated with GA-responder or GA-non-responder can be identified, for example, by genotyping a large population and performing statistical analysis to determine which alleles appear significantly more commonly together than expected. Linkage disequilibrium between two polymorphic markers or alleles is a meta-stable state. Absent selective pressure or the sporadic linked reoccurrence of the underlying mutational events, the alleles will eventually become disassociated by chromosomal recombination events and will thereby tend to reach linkage equilibrium through the course of human evolution. Thus, the likelihood of finding a polymorphic allele in linkage disequilibrium with a disease or condition may increase with changes in at least two factors: decreasing physical distance between the polymorphic allele and the condition or disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic alleles can be relied upon as an indicator of inheritance of a linked disease or condition-causing mutation, i.e., GA-responsiveness. One of skill in the art would be able to determine additional polymorphic alleles in linkage disequilibrium with the polymorphic markers of the invention. There are numerous statistical methods to detect linkage disequilibrium, including those found in Terwilliger, Am J Hum Genet, 56:777-787 (1995); Devlin, N. et al., Genomics, 36:1-16, (1996); Lazzeroni, Am J Hum Genet, 62:159-170, (1998); Service, et al. Am J Hum Genet, 64:1728-1738 (1999); McPeek and Strahs, Am J Hum Genet, 65:858-875 (1999); and U.S. patent application Ser. No. 10/480,325, all of which are herein incorporated by reference in their entirety. The nucleic acid molecules of the invention can be double- or single-stranded. Accordingly, the invention further provides for the complementary nucleic acid strands comprising the polymorphisms listed in Tables II and III.

The invention further provides allele-specific oligonucleotides that hybridize to a gene comprising a single nucleotide polymorphism or to the complement of the gene. Such oligonucleotides will hybridize to one allele of the nucleic acid molecules described herein but not a different allele. The oligonucleotides of the invention also include probes and primers which hybridize to regions 5′ and 3′ of the polymorphism. Thus such oligonucleotides can be used to determine the presence or absence of particular alleles of the polymorphic sequences described herein.

The invention provides predictive medicine methods, which are based, at least in part, on the discovery of GA-responsive polymorphic regions which are associated with the likelihood of whether a subject with MS will respond favorably to treatment with GA. These methods can be used alone, or in combination with other predictive medicine methods. The diagnostic information obtained using the diagnostic assays described herein (singly or in combination with information of another genetic defect which contributes to the same disease, e.g., MS), may be used to identify which subject will benefit from a particular clinical course of therapy useful for preventing, treating, ameliorating, or prolonging the onset of MS in the particular subject. Clinical courses of therapy include, but are not limited to, administration of medication.

In addition, knowledge of the identity of a particular GA-responsive allele in a subject, singly, or preferably, in combination, allows customization of further diagnostic evaluation and/or a clinical course of therapy for a particular disease. For example, a subject's GA-responsive genetic profile can enable a health care provider: 1) to more efficiently and cost-effectively identify means for further diagnostic evaluation, including, but not limited to, further genetic analysis; 2) to more effectively prescribe a drug that will address the molecular basis of the disease or condition; 3) to more efficiently and cost-effectively identify an appropriate clinical course of therapy; and 4) to better determine the appropriate dosage of a particular drug or duration of a particular course of clinical therapy.

The ability to target populations expected to show the highest clinical benefit, based on the GA-responsive genetic profile, can enable: 1) the repositioning of marketed drugs, e.g., GA; 2) the rescue of drug candidates whose clinical development has been discontinued as a result of safety or efficacy limitations, which are subject subgroup-specific; 3) an accelerated and less costly development for drug candidates and more optimal drug labeling (e.g., since the use of a GA-responsive polymorph as a marker is useful for optimizing effective dose); and 4) an accelerated, less costly, and more effective selection of a particular course of clinical therapy suited to a particular subject.

These and other methods are described in further detail in the following sections.

### Pharmacogenetics of the Invention

Knowledge of the identity of the allele of one or more GA-responsive gene polymorphic regions in a subject (the CTSS, MBP, TC1U3, CD95, CD86, IL-1R1, CD80, SCYA5, MMP9, MOG, SPP1 and/or IL-121U32 genetic profile), alone or in conjunction with information of other genetic defects associated with the same disease (the genetic profile of the particular disease) allows selection and customization of the therapy, e.g., a particular clinical course of therapy, e.g., GA therapy and/or further diagnostic evaluation for a particular disease to the subject's genetic profile. For example, subjects having a specific allele of a GA-responsive gene, if those subjects are symptomatic, they may or may not respond to a GA, but may respond to another drug. Thus, generation of a GA-responsive genetic profile, (e.g., categorization of alterations in GA-responsive genes which are associated with response to glatiramer acetate), permits the selection or design of drugs that are expected to be safe and efficacious for a particular subject or subject population (i.e., a group of subjects having the same genetic alteration), as well as the selection or design of a particular clinical course of therapy or further diagnostic evaluations that are expected to be safe and efficacious for a particular subject or subject population. This information on the GA-responsive genetic profile, is useful for predicting which individuals should respond to GA, particular clinical courses of therapy, or diagnostic evaluations based on their individual GA-responsive genetic profile.

In a preferred embodiment, the GA-responsive profile is the nucleotide polymorphisms indicated in Tables II and III. Pharmacogenetic studies can be performed as taught herein.

### Diagnostic Evaluation

In one embodiment, the polymorphisms of the present invention are used to determine the most appropriate treatment evaluation and to determine whether or not a subject will benefit from further treatment. For example, if a subject has two copies of a guanine allele or the complementary cytosine at nucleotide position 51 of SEQ ID NO:2, that subject is significantly more likely to respond to GA treatment compared to a subject having any other combination of alleles at that locus.

Thus, in one embodiment, the invention provides methods for classifying a subject who is or is not likely to respond to GA-therapy comprising the steps of determining the genetic profile of the subject in one or more genes selected from the group consisting of CTSS, MBP, TCRB, CD95, CD86, IL-1R1, CD80, SCYA5, MMP9, MOG, SPP1 and IL-12RB2, comparing the subject's genetic profile to a GA responders genetic population profile and/or a GA non-responders genetic population profile, and classifying the subject based on the identified genetic profiles as a subject who is or is not a candidate for GA treatment. In one embodiment, the subject's CTSS, MBP, TCRB, CD95, CD86, IL-1R1, CD80, SCYA5, MMP9, MOG, SPP1 and IL-12RB2 genetic profile is determined by identifying the nucleotide present at the nucleotide position corresponding to position 51 of SEQ ID Nos: 1, 2, 3, 4, 5, 6, 7, 8, 9, 10, 11, 12, 14, 15, 16, 18, 20, 21 and 22.

### Clinical Course of Therapy

In another aspect, the polymorphisms of the present invention are used to determine the most appropriate clinical course of therapy for a subject who has been diagnosed with relapsing remitting MS, and will aid in the determination whether the subject will benefit from such clinical course of therapy, as determined by identification of one, or polymorphisms of the invention.

In one aspect, the invention relates to the SNPs identified as described herein, both singly and in combination, as well as to the use of these SNPs, and others in these genes, particularly those nearby in linkage disequilibrium with these SNPs, both singly and in combination, for prediction of a particular clinical course of therapy for a subject who has MS. In one embodiment, the invention provides a method for determining whether a subject will or will not benefit from a particular course of therapy by determining the presence of one, or preferably more, of the identities of the polymorphisms of the invention. For example, the determination of the polymorphisms of the invention, singly, or in combination, will aid in the determination of whether an individual will benefit GA treatment, and will aid in the determination of the likelihood of success or failure of a GA course of therapy.

For example, if a subject has two copies of a guanine allele or the complimentary cytosine allele at nucleotide position 51 of SEQ ID NO:2, that subject is significantly more likely to respond to GA treatment compared to a subject having any other combination of alleles at that loci. Therefore, that subject would be more likely to require or benefit from a clinical course of therapy utilizing GA. Depending upon the genetic identity, an appropriate clinical course of therapy for MS may include, for example, continuing or suspending a GA course of treatment.

Disease Progression and/or Disease Severity

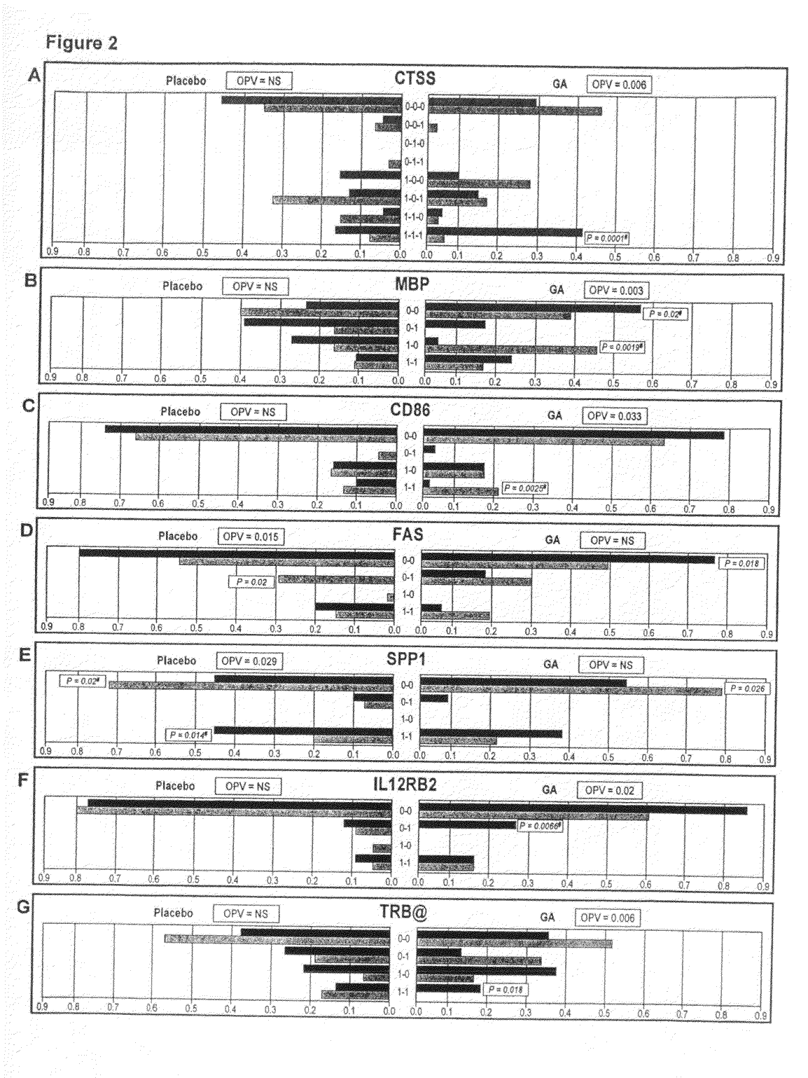

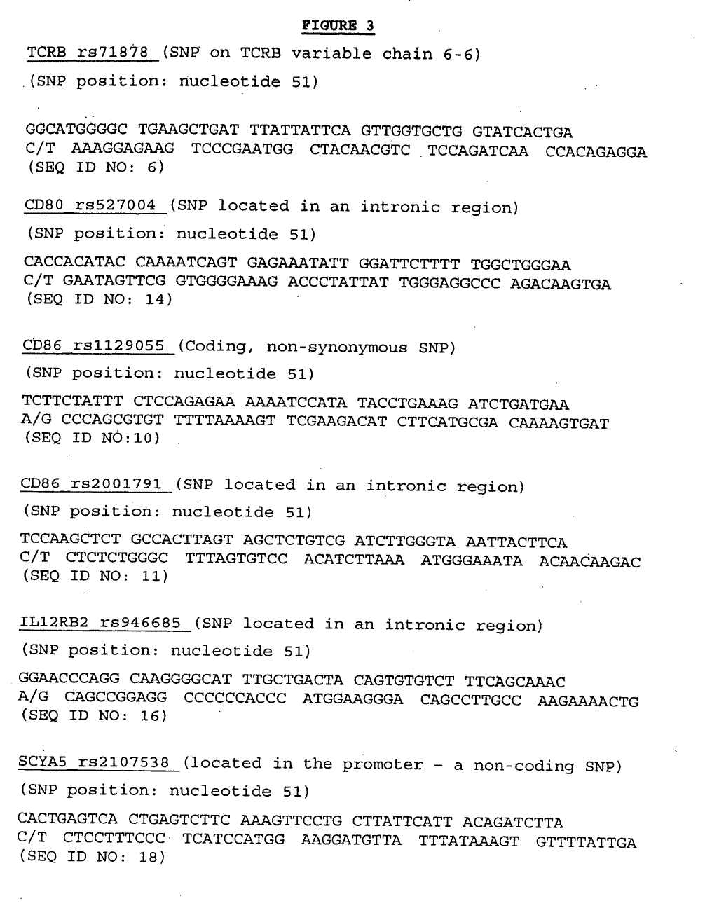

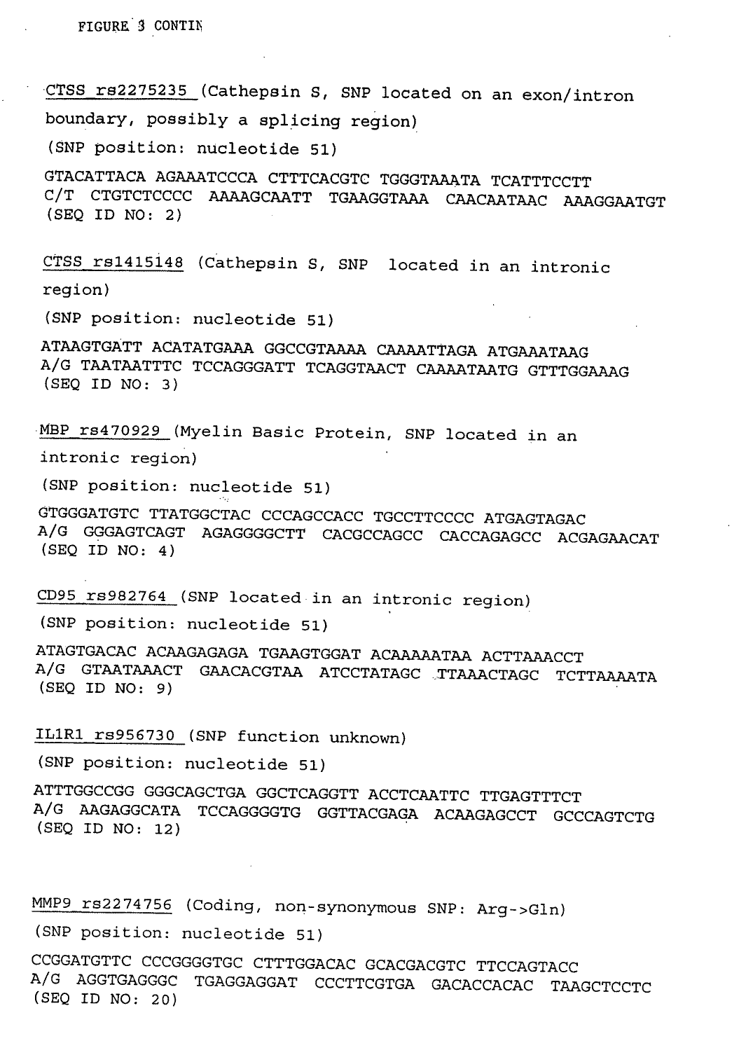

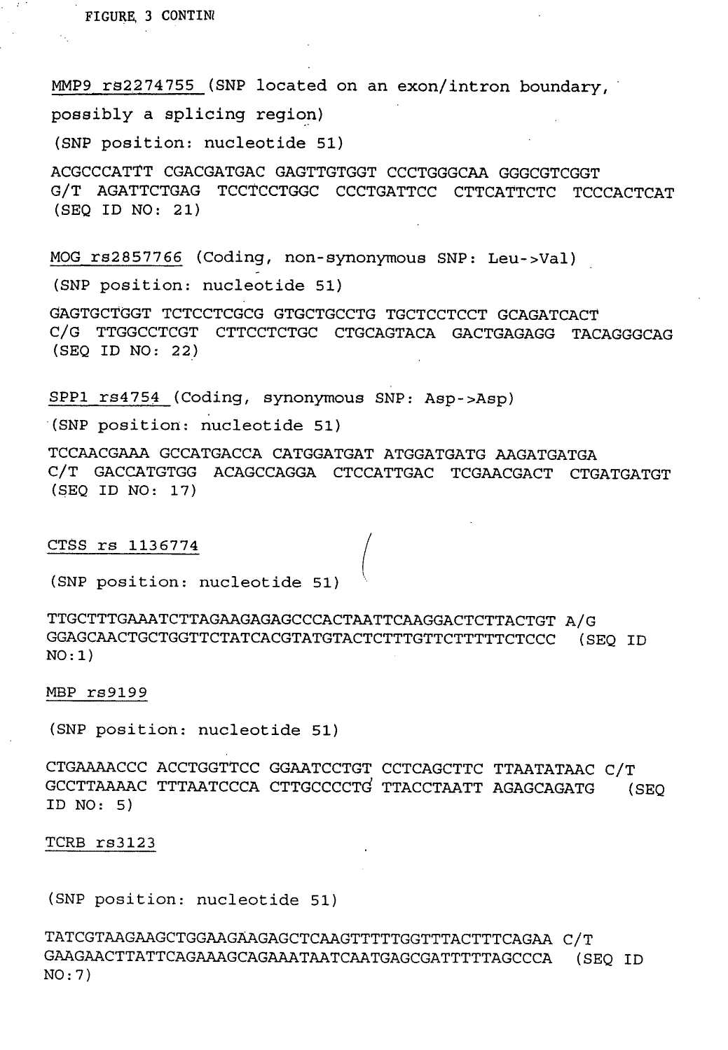

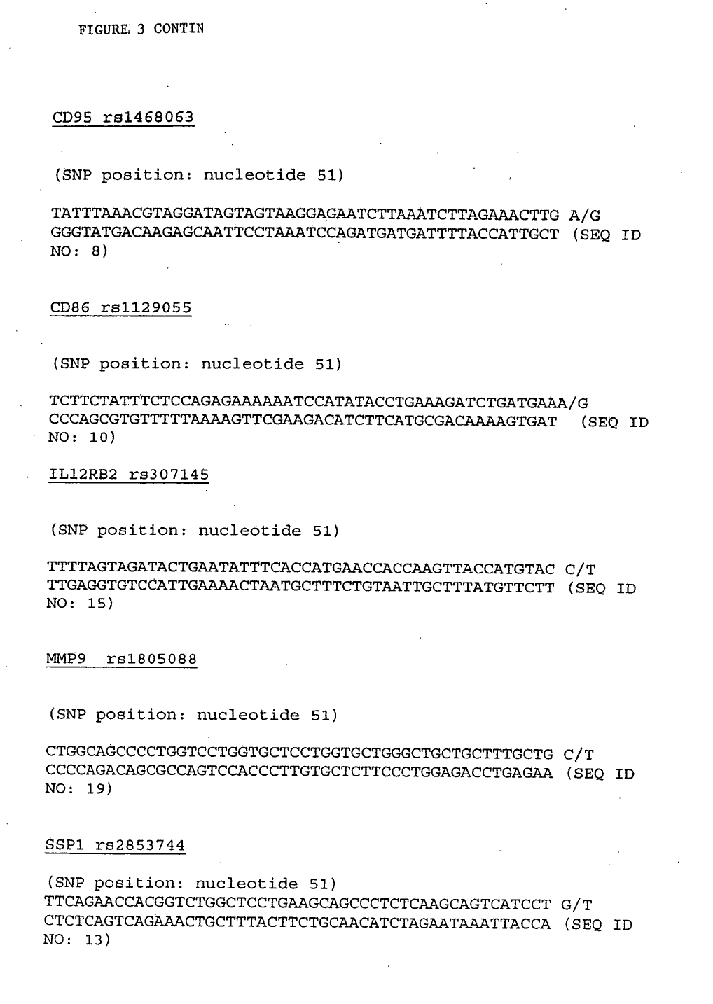

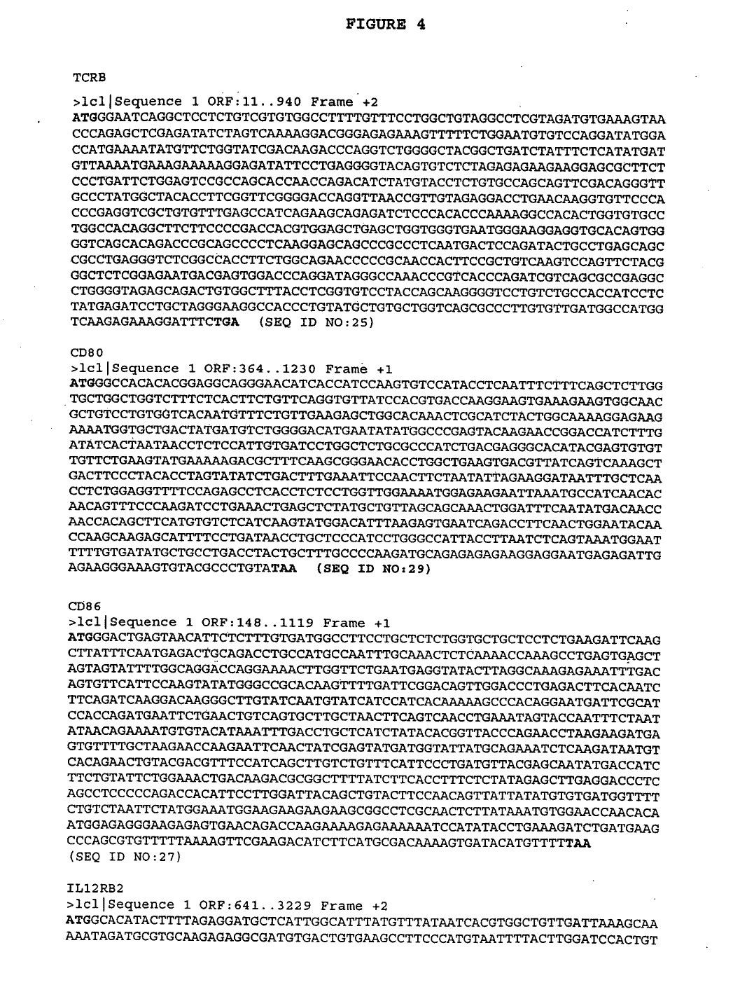

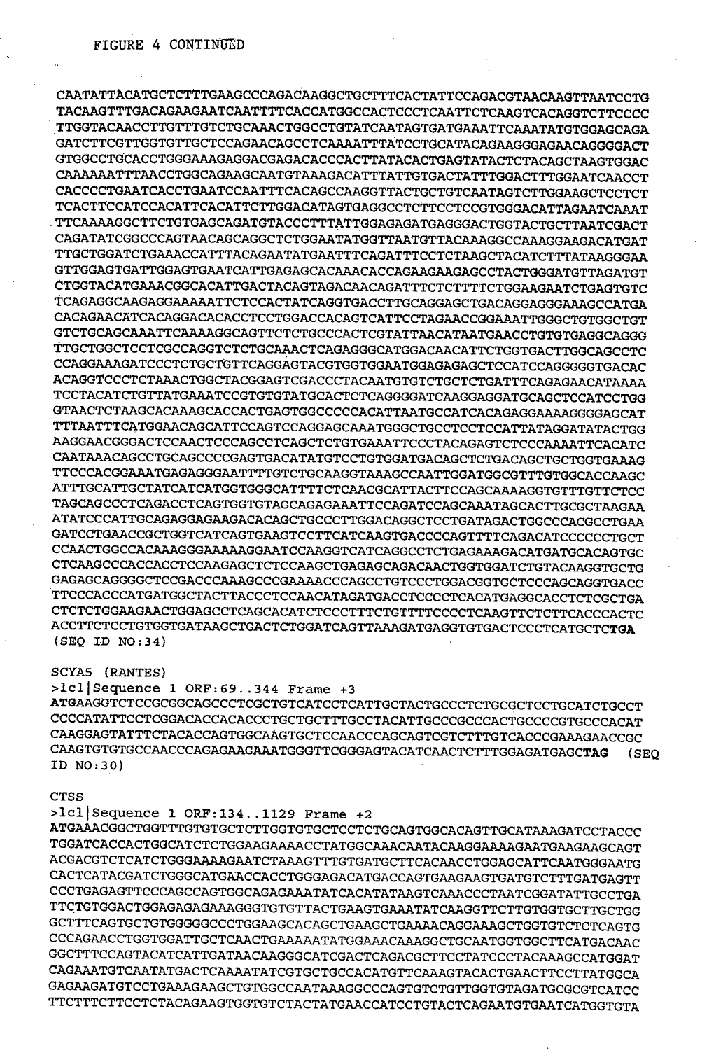

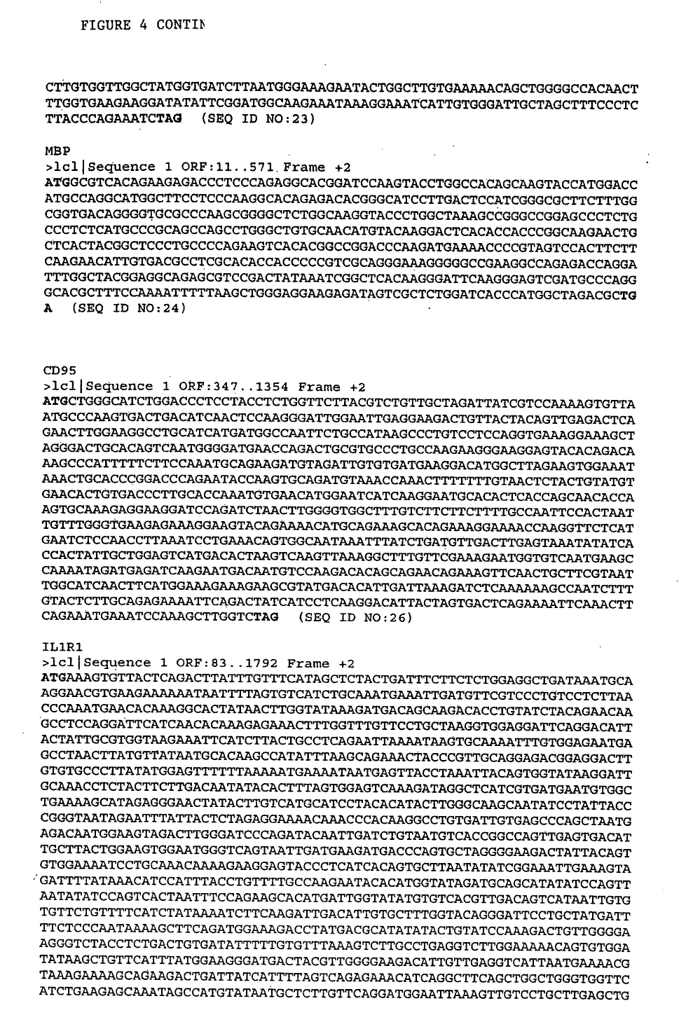

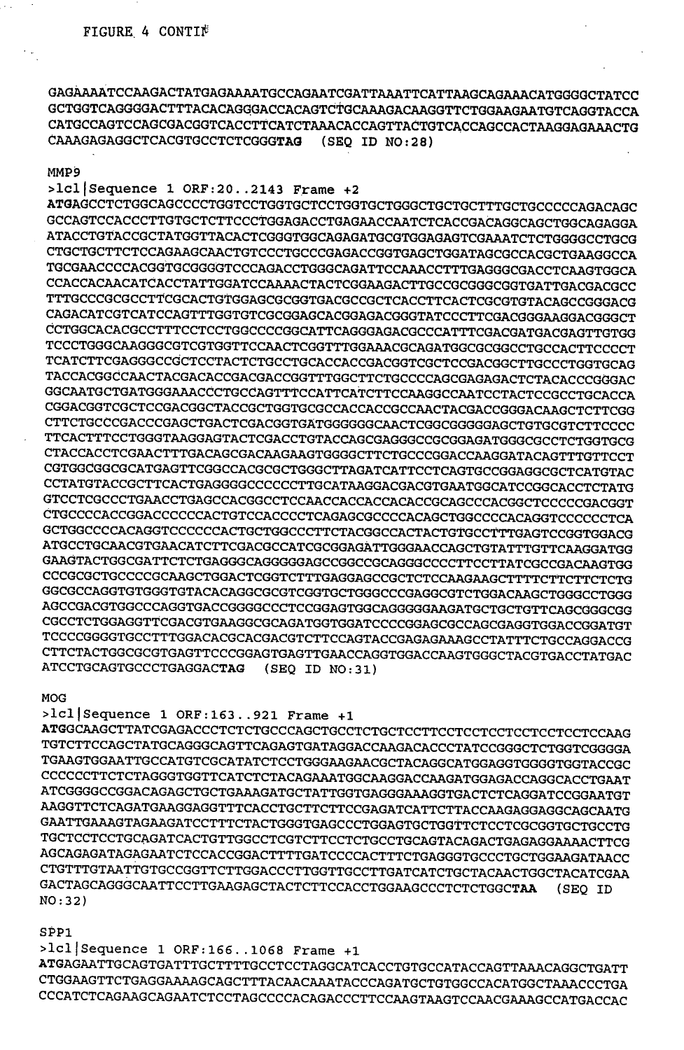

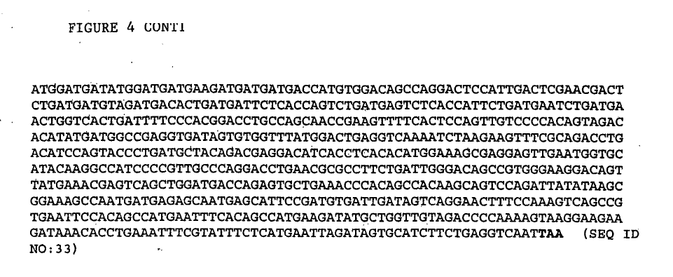

Drug-response is globally a composite mixture of drug-induced favorable effects and placebo-provoked health benefits. Large placebo effects can result in a significant loss of power, therefore placebo treated patients were analyzed to elucidate GA-induced response, distinguished from effects stemming from differential profiles of disease progression or severity. Alleles showing significant association with differential drug-response, show weaker association upon accounting for placebo effects. In addition, haplotype analysis of the SPP1 gene suggests positive correlation with response in both GA- and placebo-treated patients (FIG. 2E), due to association with differential disease severity or progression. CD95 indicates similar behavior both in haplotype analysis (FIG. 2D) and in logistic regression analysis, when conducted in the treatment groups separately, though it also shows a highly significant (p-value=0.004) association in the logistic regression comprehensive model, indicating a strong GA-response related effect. In the same context, one of the CTSS SNPs tested, rs1415148, despite being highly significant in previous analyses, had an OR of 2.5 (albeit non-significant) in the placebo-treated group. The same applies, to a lesser extent, to APOE and TCRB (data not shown), for which evidence has accumulated regarding their involvement in MS susceptibility, modifying effects and disease progression and severity. Thus, the polymorphisms of the invention are potential markers of disease progression and severity independently of their association with drug-response.

### Assays of the Invention

The present methods provide means for determining if a subject is or is not responsive to GA treatment.

The present invention provides methods for determining the molecular structure of a GA-responsive gene, such as a human CTSS, MBP, TCRB, CD95, CD86, IL-1R1, CD80, SCYA5, MMP9, MOG, SPP1 and IL-12RB2 gene, or a portion thereof. In one embodiment, determining the molecular structure of at least a portion of a CTSS, MBP, TCRB, CD95, CD86, IL-1R1, CD80, SCYA5, MMP9, MOG, SPP1 and IL-12RE32 gene comprises determining the identity of an allelic variant of at least one polymorphic region of a GA-responsive gene (determining the presence or absence of one or more of the allelic variants, or their complements). A polymorphic region of a GA-responsive gene can be located in an exon, an intron, at an introdexon border, in the 5′ upstream regulatory element, in the 3′ downstream regulatory element or in a region adjacent to a GA-responsive gene.

Analysis of one or more GA-associated polymorphic regions in a subject can be useful for predicting whether a subject is or is not likely to develop MS.

In preferred embodiments, the methods of the invention can be characterized as comprising detecting, in a sample of cells from the subject, the presence or absence of a specific allelic variant of one or more polymorphic regions of a GA-responsive gene or genes. Preferably, the presence of the variant allele of the GA-responsive gene and/or the reference allele of the GA-responsive gene described herein are detected.

In one preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip” Oligonucleotides can be bound to a solid support by a variety of processes, including lithography. For example a chip can hold up to 250,000 oligonucleotides (Genechip, Affymetrix®). Mutation detection analysis using these chips comprising oligonucleotides, also termed “DNA probe arrays” is described e.g., in Cronin et al., Human Mutation 7:244 (1996). In one embodiment, a chip comprises all the allelic variants of at least one polymorphic region of a GA-responsive gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a GA-responsive gene prior to identifying the allelic variant. Amplification can be performed, e.g., by PCR and/or LCR (See, Wu and Wallace, Genomics 4:560 (1989), according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli, J. C. et al., Proc. Natl. Acad. Sci. USA 87: 1874-1878 (1990), transcriptional amplification system (Kwoh, D. Y. et al., Proc.Natl. Acad. Sci. USA 86:1173-1177 (1989), Q-Beta Replicase (Lizardi, P. M. et al., Bio/Technology 6:1197 (1988), and self-sustained sequence replication (Guatelli et al., Proc. Nut. Acad. Sci. 87:1874 (1989), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a GA-responsive gene and detect allelic variants, e.g., mutations, by comparing the sequence of the sample sequence with the corresponding reference (control) sequence. Exemplary sequencing reactions include those based on techniques developed by Maxarn and Gilbert, Proc. Natl. Acad Sci USA 74:560 (1977) or Sanger (Sanger et al. Proc. Nut. Acad. Sci. 74:5463 (1977). It is also contemplated that any of a variety of automated sequencing procedures may be utilized when performing the subject assays (Biotechniques 19:448 (1995)), including sequencing by mass spectrometry (see, for example, U.S. Pat. No. 5,547,835 and international patent application Publication Number WO 94116101, entitled DNA Sequencing by Mass Spectrometry by H. Koster; U.S. Pat. No. 5,547,835 and international patent application Publication Number WO 9412 1822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster; Cohen et al., Adv Chromatogr 36:127-162 (1996); and Griffin, et al., Appl Biochem Biotechnol 38: 147-159 (1993). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

In some cases, the presence of a specific allele of a GA-responsive gene in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al., Science 230:1242 (1985)). In general, the technique of “mismatch cleavage” starts by providing heteroduplexes formed by hybridizing a control nucleic acid, which is optionally labeled, e.g., RNA or DNA, comprising a nucleotide sequence of a GA-responsive allelic variant with a sample nucleic acid, e.g., RNA or DNA, obtained from a tissue sample. The double-stranded duplexes are treated with an agent which cleaves single-stranded regions of the duplex such as duplexes formed based on base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al., Proc. Natl. Acad Sci USA 85:4397 (1988); Saleeba, et al., Methods Enzymol. 217:286-295 (1992). In a preferred embodiment, the control or sample nucleic acid is labeled for detection.

In another embodiment, an allelic variant can be identified by denaturing high-performance liquid chromatography (DHPLC) (Oeher and Underhill, Am. J. Human Gen. 57:Supp1. A266 (1995)). DHPLC uses reverse-phase ion-pairing chromatography to detect the heteroduplexes that are generated during amplification of PCR fragments from individuals who are heterozygous at a particular nucleotide locus within that fragment (Oefher and Underhill, Am. J. Human Gen. 57:Suppl. A266 (1995)). In general, PCR products are produced using PCR primers flanking the DNA of interest. DHPLC analysis is carried out and the resulting chromatograms are analyzed to identify base pair alterations or deletions based on specific chromatographic profiles (see O'Donovan, et al., Genomics 52:44-49 (1998)).

In other embodiments, alterations in electrophoretic mobility is used to identify the type of GA-responsive allelic variant. For example, single strand conformation polymorphism (SSCP) may be used to detect differences in electrophoretic mobility between mutant and wild type nucleic acids (Orita, et al., Proc Natl. Acad. Sci. USA, 86:2766 (1989), see also Cotton, Mutat Res., 285: 125-144 (1993); and Hayashi, Genet Anal Tech App, 19:73-79 (1992)). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen, et al., Trends Genet, 75 (1991)).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers, et al., Nature, 3 13:495 (1985)). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA Posenbaum and Reissner Biophys Chem, 265:1275 (1987).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic marker is placed centrally (allele-specific probes) and then hybridized to target DNA under conditions which permit hybridization only if a perfect match is found (Saiki et al., Nature, 324:163 (1986); Saiki et al., Proc. Natl. Acad. Sci. USA, 86:6230 (1989); and Wallace, et al., Nucl. Acids Res., 6:3543 (1979)). Such allele specific oligonucleotide hybridization techniques may be used for the simultaneous detection of several nucleotide changes in different polymorphic regions of a GA-responsive gene. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs, et al. Nucleic Acids Res., 17:2437-2448 (1989)) or at the extreme 3′ end of one primer where, under appropriate conditions, mismatch can prevent, or reduce polymerase extension (Prossner, Tibiech, 11:238 (1993); Newton, et al., Nucl. Acids Res., 17:2503 (1989)). This technique is also termed “PROBE” for Probe Oligo Base Extension. In addition it may be desirable to introduce a novel restriction site in the region of the mutation to create cleavage-based detection (Gasparini, et al., Mol. Cell. Probes, 6:1 (1992)).

In another embodiment, identification of the allelic variant is carried out using an oligonucleotide ligation assay (OLA), as described, e.g., in U.S. Pat. No. 4,998,617 and in Landegren, U. et al., Science, 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (USA), 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a GA-responsive gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe, et al., Nucleic Acids Res., 24:3728 (1996), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a GA-responsive gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91102087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA@ is described by Goelet, P. et al. (PCT Appln. No. 92/115712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/102087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res., 17:7779-7784 (1989); Sokolov, B. P., Nucl. Acids Res., 18:3671 (1990); Syvanen, A. C., et al., Genomics, 8:684-692 (1990); Kuppuswamy, M. N. et al., Proc. Natl. Acad. Sci. (USA), 88:1143-1 147 (1991); Prezant, T. R. et al., Hum. Mdat., 1:159-164 (1992); Ugozzoli, L. et al., GATA, 9:107-112 (1992); Nyren, P. et al., Anal. Biochem., 208:171-175 (1993)). These methods differ from GBAO in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., Amer. J. Hum. Genet., 52:46-59 (1993)).

For determining the identity of the allelic variant of a polymorphic region located in the coding region of a GA-responsive gene, yet other methods than those described above can be used. For example, identification of an allelic variant which encodes a mutated GA-responsive protein can be performed by using an antibody specifically recognizing the mutant protein in, e.g., immunohistochemistry or immunoprecipitation. Antibodies to wild-type GA-responsive or mutated forms of GA-responsive proteins are known in the art and can be prepared according to methods known in the art.

Alternatively, one can also measure the activity of a GA-responsive protein, such as binding to a GA-responsive ligand. Binding assays are known in the art and involve, e.g., obtaining cells from a subject, and performing binding experiments with a labeled ligand, to determine whether binding to the mutated form of the protein differs from binding to the wild-type of the protein.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA.

The methods described herein may be performed, for example, by utilizing prepackaged diagnostic kits, such as those described herein, comprising at least one probe or primer nucleic acid described herein, which may be conveniently used, e.g., to determine whether a subject is or is not likely to respond to GA-treatment, associated with a specific GA-responsive allelic variant.

Sample nucleic acid sequences to be analyzed by any of the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91107660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

### Polymorphisms Useful in the Methods of the Invention

The nucleic acid molecules of the present invention include specific allelic variants of the GA responsive genes or at least a portion thereof, having a polymorphic region. The preferred nucleic acid molecules of the present invention comprise GA-responsive sequences having one or more of the polymorphisms shown in Tables II and III. The invention further comprises isolated nucleic acid molecules complementary to nucleic acid molecules the polymorphisms of the present invention. Nucleic acid molecules of the present invention can function as probes or primers, e.g., in methods for determining the allelic identity of a GA-responsive gene polymorphic region. The nucleic acids of the invention can also be used, singly, or, preferably, in combination, to determine whether a subject is likely or unlikely to respond to GA for the treatment of MS.

As described herein, allelic variants that correlate with GA-response have been identified. The invention is intended to encompass these allelic variants. The invention also provides isolated nucleic acids comprising at least one polymorphic region of a GA-responsive gene having a nucleotide sequence which correlates with GA-responsiveness. Preferred nucleic acids used in combination in the methods of the invention to predict the likelihood of a subject diagnosed with MS to respond to GA treatment are indicated in Tables II and III.

The nucleic acid molecules of the present invention can be single stranded DNA (e.g., an oligonucleotide), double stranded DNA (e.g., double stranded oligonucleotide) or RNA. Preferred nucleic acid molecules of the invention can be used as probes or primers. Stringent conditions vary according to the length of the involved nucleotide sequence but are known to those skilled in the art and can be found or determined based on teachings in Current Protocols in Molecular Biology, Ausubel, et al., eds., John Wiley &Sons, Inc. (1995), sections 2, 4 and 6. Additional stringent conditions and formulas for determining such conditions can be found in Molecular Cloning. A Laboratory Manual, Sambrook, et al., Cold Spring Harbor Press, Cold Spring Harbor, N.Y. (1989), chapters 7, 9 and 11. A preferred, non-limiting example of stringent hybridization conditions for hybrids that are at least base-pairs in length includes hybridization in 4× sodium chloride-sodium citrate (SSC), at about 65-70° C. (or hybridization in 4×SSC plus 50% formamide at about 42-50° C.) followed by one or more washes in 1×SS C, at about 65-70° C. A preferred, non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SS C, at about 65-70° C. A preferred, non-limiting example of reduced stringency hybridization conditions for such hybrids includes hybridization in 4×SS C, at about 50-60° C. (or alternatively hybridization in 6×SSC plus 50% formamide at about 40-45° C.) followed by one or more washes in 2×SS C, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH2P04, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (T m) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=8 1 .5+16. 6(log 1 Owa+D+0.41(% G+C)−(600/N), where N is the number of bases in the hybrid, and [Na+] is the concentration of sodium ions in the hybridization buffer ([Na+] for 1×SSC=O. 165 M). It will also be recognized by the skilled practitioner that additional reagents may be added to hybridization and/or wash buffers to decrease nonspecific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non limiting example of stringent hybridization conditions is hybridization in 0.25-0.5M NaH 2P04, 7% SDS at about 65″C., followed by one or more washes at 0.02M NaH2P04, 1% SDS at 65° C., see, e.g., Church and Gilbert, Proc. Natl. Acad. Sci. USA, 81:1991-1995 (1984), (or alternatively 0.2×SS C, 1% SDS).

A primer or probe can be used alone in a detection method, or a primer can be used together with at least one other primer or probe in a detection method. A probe is a nucleic acid which specifically hybridizes to a polymorphic region of a GA-responsive gene, and which by hybridization or absence of hybridization to the DNA of a subject or the type of hybrid formed will be indicative of the identity of the allelic variant of the polymorphic region of the GA-responsive gene.

Numerous procedures for determining the nucleotide sequence of a nucleic acid molecule, or for determining the presence of mutations in nucleic acid molecules include a nucleic acid amplification step, which can be carried out by, e.g., polymerase chain reaction (PCR). Accordingly, in one embodiment, the invention provides primers for amplifying portions of a GA-responsive gene, such as portions of exons and/or portions of introns. In a preferred embodiment, the exons and/or sequences adjacent to the exons of the human GA-responsive gene will be amplified to, e.g., detect which allelic variant, if any, of a polymorphic region is present in the GA-responsive gene of a subject. Preferred primers comprise a nucleotide sequence complementary to a specific allelic variant of a GA-responsive polymorphic region and of sufficient length to selectively hybridize with a GA-responsive gene. In a preferred embodiment, the primer, e.g., a substantially purified oligonucleotide, comprises a region having a nucleotide sequence which hybridizes under stringent conditions to about 6,8, 10, or 12, preferably 25, 30, 40, 50, or 75 consecutive nucleotides of a GA-responsive gene. In an even more preferred embodiment, the primer is capable of hybridizing to a GA-responsive nucleotide sequence or complements thereof and distinguishing between a nucleotide associated with one allelic variant but not another allelic variant. For example, primers comprising a nucleotide sequence of at least about 15 consecutive nucleotides, at least about 25 nucleotides or having from about 15 to about 20 nucleotides set forth in any of SEQ ID Nos: 1, 2, 3, 4, 5, 6, 7, 8, 9, 10, 11, 12, 14, 15, 16, 18, 20, 21 and 22 or complements thereof are provided by the invention. Primers having a sequence of more than about 25 nucleotides are also within the scope of the invention. Preferred primers of the invention are primers that can be used in PCR for amplifying each of the polymorphic regions of the GA-responsive gene.

Primers can be complementary to nucleotide sequences located close to each other or further apart, depending on the use of the amplified DNA. For example, primers can be chosen such that they amplify DNA fragments of at least about 10 nucleotides or as much as several kilobases.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of the nucleotide sequence indicated in Table II and III (e.g., SEQ ID Nos: 1, 2, 3, 4, 5, 6, 7, 8, 9, 10, 11, 12, 14, 15, 16, 18, 20, 21 and 22). A reverse primer can be a primer having a nucleotide sequence or a portion of the nucleotide sequence that is complementary to a nucleotide sequence indicated in Tables II and III (e.g., SEQ ID Nos: 1, 2, 3, 4, 5, 6, 7, 8, 9, 10, 11, 12, 14, 15, 16, 18, 20, 21 and 22).

Yet other preferred primers of the invention are nucleic acids which are capable of selectively hybridizing to an allelic variant of a polymorphic region of a GA-responsive gene. Thus, such primers can be specific for a GA-responsive gene sequence, so long as they have a nucleotide sequence which is capable of hybridizing to a GA-responsive gene. Preferred primers are capable of specifically hybridizing to any of the allelic variants listed in Tables II and III. Such primers can be used, e.g., in sequence specific oligonucleotide priming as described herein.

Other preferred primers used in the methods of the invention are nucleic acids which are capable of hybridizing to and distinguishing between different allelic variants of a GA-responsive gene. Such primers can be used in combination.

The GA-responsive nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a polymorphic region of a GA-responsive gene which is polymorphic. In an even more preferred embodiment of the invention, the probes are capable of hybridizing specifically to one allelic variant of a GA-responsive gene as indicated in Tables II and III, but not other allelic variants. Such probes can then be used to specifically detect which allelic variant of a polymorphic region of a GA-responsive gene is present in a subject. The polymorphic region can be located in the 5′ upstream regulatory element, exon, or intron sequences of a GA-responsive gene.

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some pharmacogenetics methods, a portion of a GA-responsive gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, so as to be more stable than naturally occurring nucleotides. Exemplary nucleic acid molecules which are modified include phosphoramidate, phosphothioate and methylphosphonate analogs of DNA (see also U.S. Pat. Nos. 5,176,996; 5 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., Proc. Natl. Acad. Sci. USA, 86:6553-6556 (1989); Lemaitre et al., Proc. Natl. Acad. Sci. USA, 84:648-652 (1987); PCT Publication No. WO 88/09810, published Dec. 15, 1988), hybridization-triggered cleavage agents. (See, e.g., Krol et al., BioTechniques, 6:958-976 (1988)) or intercalating agents (See, e.g., Zon, Pharm. Res., 5:539-549 (1988)). To this end, the nucleic acid of the invention may be conjugated to another molecule, e.g., a peptide, hybridization triggered cross-linking agent, transport agent, hybridization-triggered cleavage agent, etc.

The isolated nucleic acid comprising a GA-responsive gene intronic sequence may comprise at least one modified base moiety which is selected from the group including but not limited to 5-fluorouracil, 5-bromouracil, 5-chlorouracil, 5-iodouracil, hypoxanthine, xanthine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual units, the strands run parallel to each other. Gautier et al., Nucl. Acids Res., 15:6625-6641 (1987). The oligonucleotide is a 2′-0-methylribonucleotide (Inoue et al., Nucl. Acids Res., 15:6131-6148 (1987)), or a chimeric RNA-DNA analogue (Inoue et al., FEBS Lett., 21 5:327-330 (1987)).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. ((1988) Nucl. Acids Res. 16:3209), methylphosphonate oligonucleotides can be prepared by use of controlled pore glass polymer supports (Sarin et al., Proc. Natl. Acad. Sci. USA, 85:7448-745(I) (1988)), etc.

The invention also provides vectors and plasmids comprising the nucleic acids of the invention. For example, in one embodiment, the invention provides a vector comprising at least a portion of a GA-responsive gene comprising a polymorphic region. The GA-responsive gene or polymorphic region can be expressed in eukaryotic cells, e.g., cells of a subject, or in prokaryotic cells.

Other aspects of the invention are described below or will be apparent to one of skill in the art in light of the present disclosure

TABLE I

| | European/Canadian trial | | | | U.S. pivotal trial | | | |
|---|---|---|---|---|---|---|---|---|
| | General* | | PGx Cohort | | General* | | PGx Cohort | |
| | Cohort (N-138) | | (N-101) | | Cohort (N-178) | | (N = 73) | |
| Variable | Mean | Std | Mean | Std | Mean | Std | Mean | Std |
| Age at First | 27.23 | 6.94 | 26.56 | 7.07 | 27.44 | 6.32 | 28.15 | 6.05 |
| Symptoms | | | | | | | | |
| Disease | 59.08 | 44.37 | 57.44 | 48.49 | 84.14 | 60.00 | 81.20 | 59.04 |
| Duration (M) | | | | | | | | |
| Relapses (2 | 2.64 | 1.59 | 2.55 | 1.7 | 2.93 | 1.27 | 2.89 | 0.99 |
| Y Prior) | | | | | | | | |
| BL EDSS | 2.32 | 1/11 | 2.30 | 1.24 | 2.66 | 1.21 | 2.52 | 1.34 |
| Score | | | | | | | | |
| BL | 1/19 | 0.98 | 1.09 | 1.01 | 1.26 | 0.99 | 0.86 | 0.82 |
| Arnbulation | | | | | | | | |
| Index | | | | | | | | |
| BL Sum FS | 4.41 | 2.83 | 4.37 | 3.31 | 5.87 | 3.46 | 5.48 | 3.75 |
| BL No. of | 4.89 | 7.04 | 3.50 | 4.13 | NA | NA | NA | NA |
| T1 lesions | | | | | | | | |
| BL T1 | 782.4 | 2082.6 | 472.6 | 610.5 | NA | NA | NA | NA |
| lesion | | | | | | | | |
| volume | | | | | | | | |
| BL New T2 | 1.06 | 1.71 | 1.12 | 1.45 | NA | NA | NA | NA |
| lesions | | | | | | | | |

TABLE II

| | | | | | | Polymorphic | | |
|---|---|---|---|---|---|---|---|---|
| | | | | | | Markers | | Nt. Position in |
| | | | | | Polymorphic | associated with | Reference | Reference |
| | SEQ ID | | | SEQ ID | Markers | GA- non- | Sequence | Sequence |
| | NO OF | Chromosome | dbSNP ID | NO. FOR | associated with | responders | GenBank | Corresponding to |
| Gene | ORF | Location | (NCBI) | SNP | GA responders | (AA changes) | Accession # | polymorphism |
| SPP 1 | 33 | 4q22.1 | rs4754 | 17 | T | | GI:28146097 | 405 |
| TCRB | 25 | 7q34 | rs71878 | 6 | C | | GI:1552506 | 27,091 |
| CD80 | 29 | 3q13.32 | rs527004 | 14 | | T | GI:19033385 | 92,290 |
| GD86 | 27 | 3q21.1 | rs1129055 | 10 | | G | GI:16572839 | 86,466 |
| | | | rs2001791 | 11 | T | | GI16572839 | 112,096 |
| IL1R1 | 28 | 2q12 | rs956730 | 12 | A | | GI:19033951 | 94,170 |
| IL12RB2 | 34 | 1p31.2 | rs946685 | 16 | G | | GI:11990046 | 103,042 |
| SCYA5 | 30 | 17q21.1 | rs2107538 | 18 | T | | GI8719360 | 168,416 |
| CTSS | 23 | 1q21.3 | rs2275235 | 2 | G | | GI21530888 | 110,515 |
| | | | rs1415148 | 3 | A | | GI21530888 | 85,991 |
| MBP | 24 | 18q23 | rs470929 | 4 | A | | GI27764783 | 128,344 |
| MMP9 | 31 | 20q13.12 | rs2274755 | 21 | | T (splice) | GI:4826835 | 669 |
| | | | rs2274756 | 20 | | A (gln) | GI:4826835 | 2022 |
| MOG | 32 | 6p21.1 | rs8257766 | 22 | | C (Val) | GI:45545416 | 673 |

TABLE III

| | | | | | | | Polymorphic | | Nt. Position in |
|---|---|---|---|---|---|---|---|---|---|
| | | | | | | Non- | Markers at Nt | Reference | Reference |
| | SEQ ID | | | SEQ ID | Responder | Responder | 51 of Respective | Sequence | Sequence |
| | NO of | Chomosome | dbSNP ID | NO For | Haplotype | Haplotype | SEQ ID | GenBank | Corresponding to |
| Gene | ORF | Location | (NCBI) | SNP | Code | Code | (AA changes) | Accession | Polymorphism |
| TCRB | 25 | 7q34 | Rs71878, | 6, 7 | 1-1 | | C-C | GI:1552506 | 27,091 |
| | | | rs3123 | | | | | GI:1552506 | 214,464 |
| CD86 | 27 | 3q21.1 | Rs1129055, | 10, 11 | | 1-1 | A (Thr)-T | GI:16572839 | 86,4661 |
| | | | rs2001791 | | | | | GI:16572839 | 112,096 |
| CD95 | 26 | 10q24.1 | Rs1468063, | 8, 9 | 0-0 | | G-A | GI:15384622 | 168,853 |
| | | | rs982764 | | | | | GI:15384622 | 163,560 |
| IL12RB2 | 34 | 1p31.2 | Rs307145, | 15, 16 | 0-1 | | G-A | GI:22004279 | 32,778 |
| | | | rs946685 | | | | | GI:11990046 | 103,042 |
| CTSS | 23 | 1q21.3 | Rs1136774, | 1, 2, 3 | 1-1-1 | | C-C-A | GI:2153088 | 118,433 |
| | | | rs2275235, | | | | | GI:2153088 | 110,515 |
| | | | rs1415148 | | | | | GI:2153088 | 85,991 |
| MBP | 24 | 18q23 | Rs470929 | 4, 5 | 0-0 | | G-T | GI:27764783 | 128,344 |
| | | | | | | | | GI:27764783 | 97,221 |

TABLE IV

| GENE&SNP-ID | TREATMENT | GEN = 0 | GEN = 1 | GEN = 2 | P-VALUE |
|---|---|---|---|---|---|
| CD80_rs527004 | GA(N = 41) | 12/34 | 6/7 | 0/0 | .0313 |
| | | (35.29%) | 85.71% | | |
| | Placebo | 13/28 | 8/15 | 1/3 | NS |
| | N = 46) | (46.43%) | (53.33%) | (33%) | |
| CD86_rs1129055 | GA(N-35) | 3/18 | 9/15 | 1/2 | 0.0404 |
| | | (16.67%) | (60%) | (50%) | |
| | Placebo | 5/22 | 4/19 | 0/2 | NS |
| | | (22.73%) | (21.05%) | | |
| CD86_rs2001791 | GA(N = 48) | 11/39 | 5/8 | 1/1 | 0.040 |
| | | (28.21%) | 62.5%) | (100%) | |
| | Placebo | 8/34 | 2/16 | 0 | NS |
| | | (22.53%) | (12.5%) | | |
| CD95_rs9827641 | GA(N-47) | 5/24 | 5/16 | 5/.7 | 0.0307 |
| | | (22.53%) | (31.25%) | ((71.43%) | |
| | Placebo | 6/22 | 3/14 | 0/12 | NS |
| | | (27.7%) | (21.4%) | | |
| CTSS_rs2275235 | GA(N = 43) | 6/31 | 4/6 | 5/6 | 0.0008 |
| | | (19.35%) | (66.67%) | (83.33%) | |
| | Placebo | 6/30 | 2/4 | 1/7 | NS |
| | | (20%) | (50%) | (14.19%) | |
| CTSS_rs1415148 | GA(N = 47) | 3/20 | 9/21 | 5/6 | 0.0018 |
| | | (15%) | (42.86%) | (83.33%) | |
| | Placebo (N = 52) | 1/18 | 8/19 | 1/5 | NS |
| | | ((5.56%) | (27.5% | (20%) | |
| MBP_rs470929 | GA(N-32) | 2/11 | 7/16 | 5/5 | 0.0038 |
| | | (18.18%) | (43.75%) | (100%) | |
| | Placebo(N = 40) | 3/16 | 4/14 | 1/10 | NS |
| | | (18.7%) | (28.57%) | (10%) | |
| TCRB_rs71878 | GA(N = 43) | 18/29 | 9/10 | 4/4 | 0.039 |
| | | 62.07% | (90%) | (100%) | |
| | Placebo(N = 38) | 10/14 | 14/15 | 7/9 | |
| | | (71.4%) | (93.33%) | (77.78%) | |

### EXAMPLES

### Example I

### Gene and Patient Selection

Subjects were selected from two previously completed randomized, double blind, placebo-controlled, multi-centric clinical trials. In both clinical trials, patients were required to have a diagnosis of definite MS (Poser C M, Paty D W, Scheinberg L, et al. New diagnostic criteria for multiple sclerosis: guidelines for research protocols. Ann Neurol 1983; 13(3):227-31) and a relapsing-remitting course (Lublin F D, Reingold S C. Defining the clinical course of multiple sclerosis: results of an international survey. National Multiple Sclerosis Society (USA) Advisory Committee on Clinical Trials of New Agents in Multiple Sclerosis. Neurology 1996; 46(4):907-11) 85 (33.9%) out of 251 patients from the U.S. pivotal trial (Johnson K P, Brooks B R, Cohen J A, et al. Copolymer 1 reduces relapse rate and improves disability in relapsing-remitting multiple sclerosis: results of a phase 111 multicenter, double-blind placebo-controlled trial. The Copolymer 1 Multiple Sclerosis Study Group. Neurology 1995; 45(7):1268-76; Johnson K P, Brooks B R, Cohen J A, et al. Extended use of glatiramer acetate (COPAXONE) is well tolerated and maintains its clinical effect on multiple sclerosis relapse rate and degree of disability. Copolymer 1 Multiple Sclerosis Study Group. Neurology 1998; 50(3):701-8), and 108 (45.2%) out of 239 patients from the European/Canadian MRI trial (Comi G, Filippi M, Wolinsky J S. European/Canadian multicenter, double-blind, randomized, placebo-controlled study of the effects of glatiramer acetate on magnetic resonance imaging—measured disease activity and burden in patients with relapsing multiple sclerosis. European/Canadian glatiramer acetate Study Group. Ann Neurol 2001; 49(3):290-7) (including patients from Holland, Italy, Belgium, UK, Canada) consented to participate in this study. The same dosage of GA (i.e. daily 20-mg subcutaneous injection) was used in both trials. Although in the original trials patients were equally assigned to either GA- or placebo-treatment, the ratios in the current PGx study were 37:36 and 4952, for the U.S. pivotal and the European/Canadian MRI trials, respectively. Table I indicates various variables between the General Cohort study and the PGx Cohort study for both the European/Canadian trial and the U.S. pivotal trial.

The primary endpoint in the European/Canadian MRI trial was the accumulated number of TI-enhancing lesions during 9 months, and an additional inclusion criterion was used calling for the presence of at least one TI-enhancing lesion on MRI screening. The primary end-point for the U.S. pivotal trial was the annualized relapse-rate after two years of treatment.

Candidate genes were selected based on their potential involvement in (a) GA's presumed mode-of-action; or (b) in MS pathogenesis; (c) representing general immune and/or neurodegenerative-related molecules; or, (d) altered gene-expression profiles associated with MS. Genes which were indicated as candidates by more than one criterion were appointed higher priority. Thus, 27 genes were selected for analysis.

### Example II

### DNA Isolation and SNP Genotyping

DNA was isolated from 174 patients and genotyped for 63 SNPs according to previously described methods (Grossman I, Avidan N, Singer C, et al. Genomic profiling of inter-population diversity guides prioritization of candidate-genes for autoimmunity. Genes Immun 2004). Briefly, DNA was extracted from leukocytes using the Roche mammalian blood DNA isolation kit according to manufacturer's instructions. Quantification and normalization of the DNA samples were done using this system in a 96 well format using DNA OD at 260 nm and 280 nm. The DNA was normalized to 50 ng/ul in costar 96-well plates. The DNA was then re-arranged in 96-well plates according to the original electronic list provided by Covance in an electronic grid, which was then computationally imported to the Sequenom MassARRAY system.

The Sequenom MassARRAY system at the Weizmann Genome Center facility provides a genotyping platform based on primer extension coupled with mass spectrometric detection, which allows the analysis of thousands of genotypes daily. Using Matrix Assisted Laser Desorption/Ionization-Time-of-Flight (MALDI-TOF) mass spectrometry, the MassARRAY system measures target DNA associated with SNPs and other forms of genetic variation directly (Chiu and Cantor 1999; Kwok 1998). The combination of SpectroCHIP arrays with the mass spectrometry technique was used (Ross et al 2000; Ross et al 1998). The method entailed the amplification of a 100-200 by DNA region containing the SNP site in a 384-well microtiter plate format followed by primer extension reactions designed to yield allele specific products with clear differences in mass. The extended and conditioned samples were transferred (14n1) to a 384 formatted spectroc˜IPTM containing preloaded matrix and analyzed in a fully automated mode by Matrix-assisted laser desorption/ionisation-time of flight mass spectrometry (MALDI-TOF MS) (SpectroREADER, Sequenom, San Diego, Calif.) and spectra are processed using SpectroTYPER (Sequenom)

In each candidate gene 2-4 SNPs were genotyped, a total of 63 SNPs. Primers and probes were designed in multiplex format (average 4.3-fold multiplexing) using SpectroDESIGNER software (Sequenom, San Diego, Calif.). Assays were successfully designed for 87% of all SNPs initially selected for the study. The remaining 13% of SNPs failed in the primer design stage, primarily due to high repeat element contents.

### Example III

### Statistical Analysis of SNP Genotyping

The procedure used stringent definitions for response to GA therapy. In the European/Canadian MRI trial: A (“combined “)-responders were defined as having no relapses throughout a 9 month follow-up and no more than one new T1-enhancing lesion in the third trimester; non-responders were defined as having at least one relapse throughout the 9 months follow-up or more than one new TI-enhancing lesion, or both; B (to be titled “TI lesion-free” hereafter)-responders were defined as exhibiting no new T1-enhancing lesions in the third trimester, while non-responders were defined as exhibiting at least one new T1-enhancing lesion in that period. For validation purposes response was also treated as a continuous variable where number of new T1-enhancing lesions within the third trimester was analyzed as the independent variable.

In the US. pivotal clinical trial responders were defined as having no evidence of disability progression and were relapse-free throughout the trial, while non-responders were defined as having at least one relapse or/and evidence of disability progression. An increase of at least one point in the EDSS score sustained at least over 3 months was defined as disability progression (“classic”).

### Statistical Analysis

All statistical analyses were performed using SAS Genetics software V.9.1 REF, in each trial separately. The different response definitions were tested in both drug- and placebo-treated cohorts, for interaction between treatment and genotype on response-outcome. Efficacy of treatment effect was tested by a Fisher two-tailed exact test disregarding the genetic data. SNPs were tested by means of the Hardy-Weinberg test (Rollnik J D, Sindem E, Schweppe C, Malin J P. Biologically active TGF-beta 1 is increased in cerebrospinal fluid while it is reduced in serum in multiple sclerosis patients. Acta Neurol Scand 1997; 96(2): 101-5) in the study's patients and in healthy control populations (Comi G, Filippi M, Wolinsky J S. European/Canadian multicenter, double-blind, randomized, placebo-controlled study of the effects of glatiramer acetate on magnetic resonance imaging—measured disease activity and burden in patients with relapsing multiple sclerosis. European/Canadian glatiramer acetate Study Group. Ann Neurol 2001; 49(3):290-7) and thus two SNPs were excluded from further genotyping. A hierarchical analysis was employed to analyze the 63 SNPs. These SNPs were analyzed both singly and in haplotypes.

### SNP-by-SNP Analysis

Three statistical methods for testing a marker for association with GA response were employed: Armitage's trend test (Armitage P. Tests for linear trends in proportions and frequencies. Biometrics 1955(11):375-86.) the allele case-control test (Fisherian 2×2 table), and the genotype case-control test (Fisherian 3×2 table) (Sasieni P D. From genotypes to genes: doubling the sample size. Biometrics 1997; 53(4):1253-61) Monte Carlo estimates of exact p-values were computed using 100,000 permutations (Westfall P H, Young S S. Resampling-based multiple testing. New York: John Wiley & Sons, Inc.; 1993). Results from the exact Armitage trend test are presented, although most results are reproduced in all methods.

SNPs showing significant association to GA response were confirmed by a logistic regression model using all patients (GA- and placebo-treated). The model contained two independent variables: a “drug” indicator variable D (drug or placebo), the genotype variable G (having three possible values: 0 or 1 or 2) and the interaction between them (D*G), namely:

Log Odds=β₀+β₁D=β₂G+β₃D*G,

where βo is the intercept and pi (i=1 to 3) is the change in log Odds as a result of a unit increase in D, G, or D*G, respectively. Association was defined as a significant (p<0.05) drug-by-genotype interaction effect. Baseline characteristics were adjusted by covariates supplement to the model (such as gender, age, country, baseline EDSS score, number of relapses 2 years prior to trial initiation, etc.) In addition, a logistic regression model including covariates was performed separately on each cohort estimating the linear odds ratio (OR) for each SNP. A Poisson model including the same covariates and analysis of covariance (ANCOVA) were performed in order to investigate influence of baseline differences between groups. SNPs showing statistically significant associations to GA response in the European/Canadian MRI trial were analyzed by continuous variables as well (number of relapses throughout trial/number of T1-enhancing lesions in third trimester) by a Kruskall-Wallis test.

### Haplotype Analysis

Genes successfully genotyped for at least two SNPs were tested for haplotype association with response to GA. The Expectation-Maximization (EM) algorithm (Excoffier L, Slatkin M. Maximum-likelihood estimation of molecular haplotype frequencies in a diploid population. Mol Biol Evol 1995; 12(5):921-7) was used to reconstruct haplotypes and to estimate their frequencies under the assumption of HWE. Omnibus likelihood ratio tests were generated (Fallin D, Cohen A, Essioux L, et al. Genetic Analysis of Case/Control Data Using Estimated Haplotype Frequencies Application to APOE Locus Variation and Alzheimer's Disease. Genome Res 2001; 1 1(1): 143-5 I), as well as individual haplotype associations (whenever the omnibus test was significant). Empirical p-values were calculated (10,000 permutations).

Polymorphisms Associated with Responders and Non-Responders.

63 SNPs were genotyped, within the 27 selected genes, in order to uncover genetic associations with response to GA and its clinical response features. Six of the SNPs deviated significantly from Hardy-Weinberg equilibrium @WE) expectations. Out of these, two SNPs were identified at an early stage and excluded from further genotyping. The remaining four SNPs might be associated to MS disease susceptibility, rather than, or in addition to, GA-response determination, since some of them show similar HWE deviations in control populations.

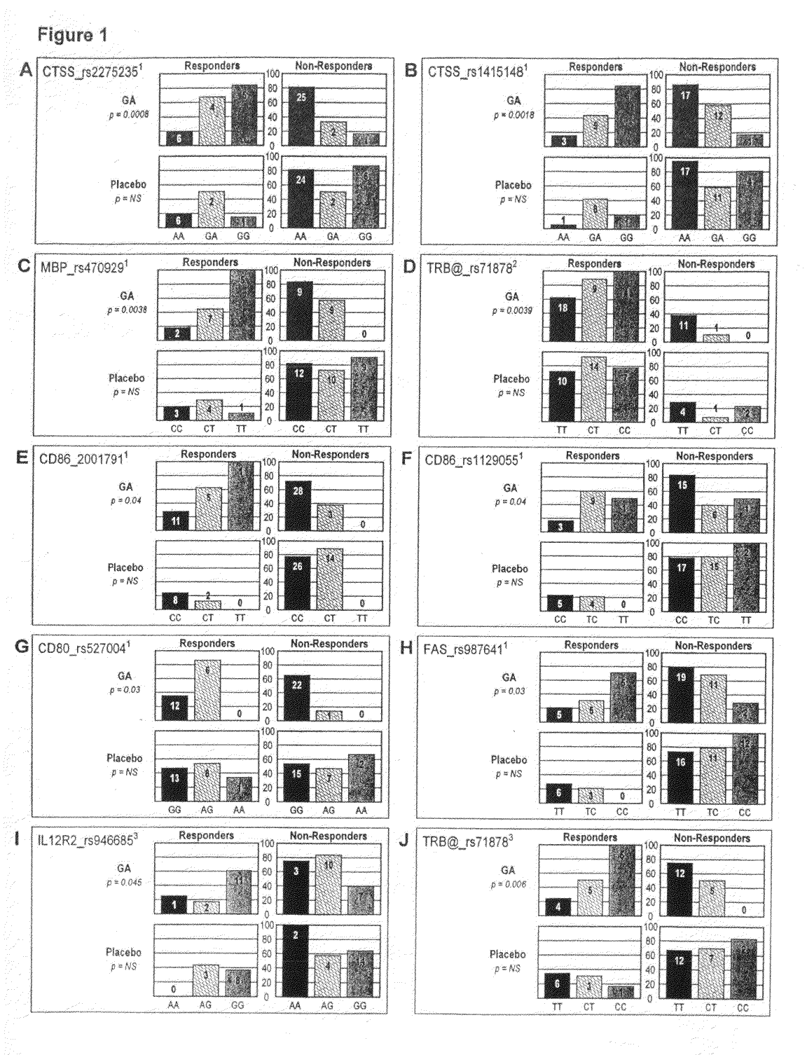

Case-control analysis of 61 SNPs in 27 genes, based on the “combined” response definition and the “TI lesion-free” response definitions, within the GA-treated group of the European/Canadian clinical trial (Table TV; FIGS. 1A-1H), identified significant associations with five genes (eight SNPs). The same analysis based on a “classic” response definition within the GA-treated group of the U.S. pivotal trial (FIGS. 11 and 1J), identified significant associations with two genes/SNPs (IL-12RB2 and TCRB). The observed differences in genotype frequencies in responders versus non-responders within the GA-treated groups were not detected in any of these SNPs in responders versus non-responders within the placebo-treated groups. Thus, these specific alleles may contribute to the drug-induced treatment response.

In the European/Canadian MRI trial these genes include Cathepsin S (CTSS), a protease crucially involved in MHC class II antigen presentation, and the main MBP-degrading enzyme; and Myelin Basic Protein (MBP), the autoantigen attacked by the immune system in Multiple Sclerosis. TCRB was implicated based on the TI-lesion free response definition (FIG. 1D; Table IV), which was further corroborated via definition of response as a continuous variable of cumulative number of TI-lesions Op=0.039). In two CTSS SNPs (rs2275235 and rs1415148) the heterozygote has about twice the likelihood to respond, and the homozygote for the response allele four times as much, than that of the homozygote for the null allele (FIGS. 1A and 1B; Table N). Similar pattern can also be observed in MBP, and to a lesser extent in TCRB.

Haplotype frequency analysis resulted in statistically significant associations between GA-response, “combined” definition, and five genes: CD86, MBP, CD95, CTSS and SPP1 in the European/Canadian MRI trial (FIG. 2A-2E). The differences in haplotype frequencies between responders and non-responders are indeed further enhanced as opposed to genotype 5 distribution analysis. For example, in both CTSS and MBP a single haplotype, 1-1-1 and 1-0 respectively, is the major haplotype in non-responders, accounting for 40-50% of subjects. In contrast, these haplotypes' frequencies in responders are less then 5%. This difference in haplotype frequency is one of the largest reported in PGx studies. “Omnibus” likelihood ratio test statistic was calculated per gene as well, assessing the overall haplotype frequency profile differences between responders and non-responders (Fallin D, Cohen A, Essioux L, et al. Genetic Analysis of Case/Control Data Using Estimated Haplotype Frequencies: Application to APOE Locus Variation and Alzheimer's Disease. Genome Res 2001; 11(1):143-51). “Omnibus” p-values in the GA-treated group were 0.003 for MBP, 0.0058 for CTSS, 0.0331 for CD86, 0.0512 for CD80, 0.067 for CD95 and 0.015 for CD95 in the placebo-treated group, 0.029 for SPP1 and 0.076 for SPP1 in the placebo-treated group.

Haplotype frequency analysis resulted in statistically significant associations between GA-response, the “classic” definition, and IL-12RB2 and TCRB. “Omnibus” p-values in the GA-treated group were 0.02 for IL-12RB2 and 0.006 for TCRB. (FIGS. 2F and 2G). The results of the haplotype analysis further support the proposed PGx association of variants in these genes with GA-response in both trials.

Logistic regression analysis was also conducted. Both the GA- and placebo-treated groups were analyzed simultaneously. Significant drug-by-genotype interactions for the European/Canadian MRI trial were found for genes MBP and CTSS, which had shown significance both in the single SNPs and haplotype analysis, CD86, CD95 (FAS) and IL1R1. A significant drug-by-genotype association for the U.S. pivotal trial was detected in the TCRB gene. The impact of each genotype on the phenotype was measured by means of Odds Ratio (OR). The high and significant OR values suggest that each allele might have substantial contribution to the probability of a patient to respond to GA treatment.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1-26. (canceled)

27. A method of identifying a human subject as a responder to treatment with glatiramer acetate, the method comprising,
obtaining from the subject a nucleic acid sample; and
determining the presence in the nucleic acid sample of a polymorphic marker at a nucleotide corresponding to nucleotide position 27,091 or to nucleotide position 214,464 of Gen Bank Accession # GI:1552506 of the T-cell receptor β (TCRB) gene, or the complements thereof, wherein the presence of the polymorphic marker is indicative of the subject being a responder to glatiramer acetate.

28. The method of claim 27, wherein the polymorphic marker is a cytosine.

29. A method of identifying a responder to treatment with glatiramer acetate, the method comprising the steps of:
obtaining a nucleic acid sample from a subject having symptoms associated with a disease or condition amenable to treatment with glatiramer acetate; and
determining the presence in said nucleic acid sample of one or more polymorphic markers associated with a response to glatiramer acetate treatment, said one or more polymorphic markers occurring at a nucleotide corresponding to position 51 of a sequence selected from the group consisting of: SEQ ID NO:2, SEQ ID NO:3, SEQ ID NO:4, SEQ ID NO:6, SEQ ID NO:9, SEQ ID NO:11, SEQ ID NO:12, SEQ ID NO:16, and SEQ ID NO:18, or the complements thereof, wherein the presence of one or more polymorphic markers is indicative of a responder to glatiramer acetate.

30. The method of claim 1 wherein the disease or condition amenable to treatment with glatiramer acetate is relapsing-remitting multiple sclerosis, an inflammatory bowel disease or graft rejection.

31. A kit for identifying a responder or a non-responder to treatment with glatiramer acetate, comprising:
a) a probe or primer which detects or amplifies position 51 of a nucleic acid sequence selected from the group consisting of: SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:I0, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21 and SEQ ID NO:22.
b) means for detecting the nucleic acid sequence.
